(19) United States
(12) Patent Application Publication
(10) Pub. No.: US 2026/0165836 A1
(43) Pub. Date: Jun. 18, 2026

(54) INTRODUCER/DILATOR WITH FOLDED BALLOON

(71) Applicant: Edwards Lifesciences Corporation, Irvine, CA (US)

(72) Inventors: Tiffany Diemtrinh Tran, Anaheim, CA (US); Jeong Soo Lee, Yorba Linda, CA (US)

(73) Assignee: Edwards Lifesciences Corporation, Irvine, CA (US)

(21) Appl. No.: 19/462,927

(22) Filed: Jan. 28, 2026

Related U.S. Application Data
(63) Continuation of application No. PCT/US2024/040131, filed on Jul. 30, 2024.
(60) Provisional application No. 63/530,144, filed on Aug. 1, 2023.

(51) Int. Cl. A61F 2/24; A61M 25/10

(52) U.S. Cl. CPC A61F 2/2433; A61M 2025/1004; A61M 25/1018; A61M 2025/1075

(57) ABSTRACT

Various implementations include an inflatable sheath for delivering a medical device, the sheath includes a fluid chamber (for example, a fluid-bearing chamber) that when inflated expands the sheath from an unexpanded configuration in which the central lumen has a first diameter to an expanded configuration in which the central lumen has a second, larger, diameter.

### CROSS-REFERENCE TO RELATED APPLICATIONS

This application is a continuation of International Application No. PCT/US2024/040131, filed Jul. 30, 2024, and entitled “Introducer/Dilator with Folded Balloon,” which claims the benefit of U.S. Provisional Application No. 63/530,144, filed Aug. 1, 2023, the contents of which are incorporated herein by reference in their entirety.

### FIELD

The present application is directed to a sheath and introducer/dilator for use with catheter-based technologies for repairing and/or replacing heart valves, as well as for delivering an implant, such as a prosthetic valve to a heart via the patient's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient's vasculature (for example, the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Expandable introducer sheaths, formed of highly elastomeric materials, allow for the dilating of the vessel to be performed by the passing prosthetic device. However, portions of the sheath resist expansion requiring higher push forces for advancement of the delivery apparatus and implant to the treatment location. Increased push forces are also experienced when the sheath, delivery apparatus and/or implant encounter a narrowed or hardened blood vessel, a calcific lesion, or other blockage/occlusion that may impede delivery of the medical device to the treatment location.

Accordingly, there remains a need for systems and methods that reduce the push force needed to introduce the delivery device and implant to the treatment location within a patient's blood vessel.

### SUMMARY OF THE INVENTION

Implementations of the present expandable sheath system can minimize trauma to the vessel and damage to the sheath and prosthetic device by reducing push forces through the blood vessel and/or sheath by providing a combined introducer-dilator that can be used to dilate the blood vessel and or sheath before the delivery device/implant is introduced. Some examples can comprise a sheath with a smaller profile than that of prior art introducer sheaths. Furthermore, some examples can reduce the length of time a procedure takes, as well as reduce the risk of a longitudinal or radial vessel tear, or plaque dislodgement because lower push force is required and only one sheath is used, rather than several different sizes of sheaths and/or dilators.

An example sheath system according to the present disclosure includes: a radially expandable sheath including an inner layer and an optional tubular strain relief layer provided over the inner layer that limits radial expansion of the sheath. The system also includes an introducer dilator sized and configured to be received within the lumen of the sheath for expanding at least a portion of patient's blood vessel and/or the sheath (for example, the inner layer and/or strain relief layer of the sheath).

An example sheath system of the present disclosure includes a radially expandable sheath and a dilator sized and configured to be received within the central lumen of the sheath.

In some examples, the sheath includes a continuous inner layer defining a central lumen extending therethrough, the inner layer having at least one folded portion extending along a length of the inner layer.

In some examples, the dilator includes an elongated dilator shaft including an inflatable expansion element provided thereon where the expansion element including an inflation chamber (for example, a fluid-bearing chamber).

In some examples, upon receipt of an inflation fluid within the inflation chamber, the expansion element is movable from an unexpanded configuration in which the expansion element has a first diameter to an expanded configuration in which the expansion element has a second, larger diameter.

In some examples, upon removal of the inflation fluid from the inflation chamber the expansion element returns to the unexpanded configuration.

In some examples, in the unexpanded configuration the inflation chamber has a first volume and in the expanded configuration the inflation chamber has a second, larger, volume.

In some examples, the sheath system includes an inflation fluid (for example, a fluid (saline) and/or a gas) provided to the inflation chamber (for example, via an inflation lumen in fluid communication with an inflation port provided on the dilator hub).

In some examples, the expansion element expands from the unexpanded configuration to the expanded configuration upon receipt of the inflation fluid in the inflation chamber (for example, the expansion element expands in response to the outwardly directed radial force exerted on the inner wall of the expansion element from the increased volume of the inflation fluid received within the inflation chamber).

In some examples, the expansion element moves from the expanded configuration to the unexpanded configuration upon withdrawal of the inflation fluid from the inflation chamber (for example, the expansion element returns to the unexpanded configuration in response a reduction in the outwardly directed radial force exerted on the inner wall of the expansion element by the inflation fluid resulting from a decrease in the volume of the inflation fluid received within the inflation chamber).

In some examples, in the expanded configuration, the expansion element has a second, larger, diameter ranging from 12F to 45F (for example, the expanded outer diameter of the expansion element ranges from 4 mm to 15 mm, and the unexpanded diameter ranges from 3 mm to 6 mm).

In some examples, in the expanded configuration, the expansion element has a regular or irregular shaped (for example, radial and/or longitudinal) cross-section.

In some examples, in the expanded configuration, the expansion element has a curvilinear shaped radial and/or longitudinal cross-section.

In some examples, in the expanded configuration, the expansion element has a circular shaped radial and/or longitudinal cross-section.

In some examples, in the expanded configuration, the expansion element has a rectilinear shaped radial and/or longitudinal cross-section.

In some examples, in the expanded configuration, the expansion element has an elliptical shaped longitudinal cross-section.

In some examples, in the expanded configuration, the expansion element projects beyond an outer surface around an entire circumference of the dilator/dilator shaft.

In some examples, in the expanded configuration, the expansion element projects beyond an outer surface around a portion of an entire circumference of the dilator.

In some examples, the expansion element is composed of a semi-compliant (high-strength) material (for example, Polyether Block Amide (Pebax), high durometer polyurethanes having Shore D durometer greater than 65D, polyamide). In some examples, the expansion element is composed of a non-compliant (ultra high-strength) material (for example, polyester such as polyethylene terephthalate or polybutylene terephthalate).

In some examples, the expansion element is composed of a polyamide, co-polyamide, polyether block amide, polyurethane (PE), polyolefin, polyethylene terephthalate (PET), nylon, and/or polybutylene terephthalate.

In some examples, the expansion element resists burst pressure of at least about 5 atm.

In some examples, in the unexpanded configuration, the expansion element is folded against the dilator shaft, wherein an outer diameter of the of the expansion element in the unexpanded configuration is less than or equal to an outer diameter of the dilator.

In some examples, the dilator shaft includes a reduced diameter portion, wherein the unexpanded configuration, the expansion element is folded against the reduced diameter portion such that the outer diameter of the expansion element is not greater than an outer diameter of the dilator shaft (for example, outer surface of the dilator shaft).

In some examples, in the unexpanded configuration, the expansion element is folded against the dilator shaft in a propeller fold configuration (for example, including 5-8 folds).

In some examples, the reduced diameter portion includes a distally-tapered rear wall (for example, to provide space for the deflated expansion element to help with retrieval/removal of the dilator/expansion element from the patient's vasculature).

In some examples, the expansion element is covered by an elastomer cover.

In some examples, the elastomer cover restrains the expansion element in the unexpanded configuration and moves with the expansion element during expansion. In some examples, the elastomer cover has a low expansion force (for example, a Shore A durometer lower than 95A).

In some examples, the dilator shaft includes an elongated body portion adjacent a proximal end of the dilator shaft, and a tapered portion extending from a distal end of the dilator shaft toward the body portion (for example, the dilator/dilator shaft has a Shore D durometer ranging from 63D to 75D).

In some examples, the expansion element is provided on the body portion.

In some examples, the expansion element is provided on the body portion proximate the distal end of the dilator shaft (for example, at a location between the midpoint of the dilator shaft and the distal end of the dilator shaft).

In some examples, when the dilator is advanced (for example, fully advanced) within the central lumen of the sheath, the expansion element is located beyond the distal end (for example, distal opening) of the sheath. In some examples, in this position, the expansion element can be used to dilate the patient blood vessel, used to dilate a tight or hardened vessel, or disrupt/dislodge a calcific lesion, or other blockage/occlusion that may impede delivery of the medical device to the treatment location. In some examples, the dilator has a length ranging from 25 cm to 100 cm. In some examples, the dilator is approximately 45 cm long. In some examples, the expansion element is located 3 inches to 5 inches from the distal end of the dilator.

In some examples, the dilator further includes an inflation lumen in fluid communication with the inflation chamber for providing an inflation fluid thereto (for example, where the inflation lumen is in fluid communication with an inflation port provided on the dilator hub).

In some examples, the dilator further includes in inner surface, outer surface, and a central lumen passing therethrough. In some examples, the central lumen is sized and configured to receive a guidewire, for example, having a diameter of approximately 0.030 inches. In some examples, the central lumen is defined by the inner surface of the dilator.

In some examples, the inner surface is composed of a low friction material (for example, HDPE, fluoropolymers), a low friction coating, and/or a hydrophilic material or coating (for example, a hydrophilic polymer blend, hydrophilic coating, hydrophilically lubricious blend). In some examples, the dilator (for example, the outer layer of the dilator) is composed of nylon 12.

In some examples, the inflation lumen extends longitudinally within a wall thickness the dilator provided between the inner surface and the outer surface of the dilator.

In some examples, the inflation lumen has any regular or irregular shaped cross-section.

In some examples, the inflation lumen has a circular shaped cross-section.

In some examples, the inflation lumen has a crescent shaped cross-section.

In some examples, the inflation lumen has an annular shaped cross-section.

In some examples, at least a portion of the sheath is configured to locally expand from an unexpanded configuration in which the central lumen has a first diameter to an expanded configuration in which the central lumen has a second, larger, diameter (for example, due to the outwardly directed radial force exerted on the lumen of the inner layer by the dilator and/or a medical device against the inner layer), and then locally contract at least partially back to the unexpanded configuration as the outwardly directed radial force is removed (for example, as the dilator and/or medical device passes through the lumen).

In some examples, the sheath further includes an outer layer provided over the inner layer, where the outer layer is discontinuous and includes an overlapping portion and an underlying portion, and the overlapping portion overlaps the underlying portion. In some examples, at least a portion of the folded portion of the inner layer is positioned between the overlapping and underlying portions, a tubular strain relief layer provided over the inner layer that limits radial expansion of the sheath, where the strain relief layer extends at least partially over the outer layer.

In some examples, when the sheath is in the unexpanded configuration, the folded portion extends circumferentially over an outer surface of the inner layer and/or outer layer.

In some examples, when the sheath is in the expanded configuration, local expansion causes a length of the folded portion to at least partially unfold.

In some examples, when the sheath is in the expanded configuration, local expansion of the sheath causes a length of the overlapping portion to move circumferentially with respect to the underlying portion.

In some examples, when the sheath is in the expanded configuration, local expansion of the sheath forms a gap between longitudinally extending edges of the outer layer, where at least a portion of the unfolded portion extends into the gap.

In some examples, the central lumen of the inner layer is cylindrical in the nonexpanded and expanded configurations.

In some examples, the inner layer comprises PTFE and the outer layer comprises IDPE and/or Tecoflex.

In some examples, the inner and outer layers are bonded together.

In some examples, the inner and outer layers are thermally bonded together.

In some examples, the inner and outer layers are bonded together by an adhesive.

In some examples, the strain relief layer is bonded to the outer layer and/or inner layer.

In some examples, the strain relief layer is thermally and/or adhesively bonded to the outer layer and/or inner layer.

In some examples, the inner layer comprises a woven fabric and/or braided filaments.

In some examples, the inner layer comprises yarn filaments of PTFE, PET, PEEK, and/or nylon.

In some examples, the outer layer comprises polyurethane (for example, high density polyethylene).

In some examples, the sheath further includes an elastic outer cover extending at least partially over the sheath (for example, at least partially over the inner layer, the outer layer, and/or the strain relief layer) where the outer cover locally expands and contracts as the medical device is advanced through the lumen.

In some examples, the elastic outer cover exerts a radially inward force on the sheath (for example, urging the inner layer, outer layer, and or strain relief layer to the unexpanded configuration).

In some examples, the elastic outer cover comprises PEBAX, polyurethane, silicone, or polyisoprene, or combination thereof.

In some examples, the sheath includes a tubular strain relief layer provided over the inner layer positioned at a proximal end of the sheath and extending along at least a portion of a length of the sheath, where the strain relief layer comprises a stiffer and/or less elastomeric material than the inner layer and/or outer layers that restricts expansion of the inner and outer layers.

In some examples, an overall length of the strain relief layer and/or sheath does not change when the sheath and/or strain relief layer moves between the unexpanded and expanded configuration.

In some examples, the strain relief layer includes a proximal portion adjacent a proximal end of the strain relief layer, a distal portion adjacent a distal end of the stain relief layer, and a tapered portion extending between the distal portion and the proximal portion, where the diameter of the proximal portion is greater than the diameter of the distal portion

In some examples, at least a portion of the strain relief layer is configured to locally expand from an unexpanded configuration at a first diameter to an expanded configuration at a second, larger, diameter (for example, in response to an outwardly directed radial force exerted on the lumen by the expansion element of the dilator and/or medical device), and then locally contract at least partially back to the unexpanded configuration (for example, as the dilator (and/or medical device) passes through the lumen).

In some examples, the strain relief layer comprises a material having a higher durometer than at least one of the inner layer (and/or the outer layer) of the sheath.

In some examples, the strain relief layer comprises polyurethane (for example, high density polyethylene).

In some examples, the dilator includes a dilator hub coupled to the proximal end of the dilator shaft, the dilator hub including an inflation port in fluid communication with the inflation lumen.

In some examples, the sheath further includes sheath hub fixedly coupled to the proximal end of the sheath, the sheath hub including a central lumen extending therethrough and coaxial with the lumen of the sheath, wherein the dilator shaft is sized and configured to be received (for example, slidably received) within the central lumen of the sheath hub.

In some examples, dilator hub is configured to be coupled to the sheath hub (for example, coupled by a press fit, an interference fit, a snap fit, a pin, thread, bayonet fastener, clip, locking key).

In some examples, the sheath hub includes one or more seals for forming a seal around an outer surface of the dilator (and/or delivery apparatus) movable through the central lumen of the sheath hub.

Another example of the present disclosure is directed to an inflatable dilator including an elongated dilator shaft sized and configured to be received within a central lumen of a sheath used for delivering a medical device; and an inflatable expansion element provided on the dilator shaft, the expansion element including an inflation chamber (for example, a fluid-bearing chamber).

In some examples, upon receipt of an inflation fluid within the inflation chamber, the expansion element is movable from an unexpanded in which the expansion element has a first diameter to an expanded configuration in which the expansion element has a second, larger diameter, and upon removal of an inflation fluid from the inflation chamber the expansion element returns to the unexpanded configuration.

In some examples, in the unexpanded configuration the inflation chamber has a first volume and in the expanded configuration the inflation chamber has a second, larger, volume.

In some examples, the dilator further includes an inflation fluid (for example, a fluid (saline), a gas) provided to the inflation chamber (for example, via an inflation lumen in fluid communication with an inflation port provided on the dilator hub).

In some examples, the expansion element expands from the unexpanded configuration to the expanded configuration upon receipt of the inflation fluid in the inflation chamber (for example, the expansion element expands in response to the outwardly directed radial force exerted on the inner wall of the expansion element from the increased volume of the inflation fluid received within the inflation chamber), and the expansion element moves from the expanded configuration to the unexpanded configuration upon withdrawal of the inflation fluid from the inflation chamber (for example, the expansion element returns to the unexpanded configuration in response a reduction in the outwardly directed radial force exerted on the inner wall of the expansion element by the inflation fluid resulting from a decrease in the volume of the inflation fluid received within the inflation chamber).

In some examples, in the expanded configuration, the expansion element has a second, larger, diameter ranging from 12 F to 45 F. In some examples, the expanded outer diameter of the expansion element ranges from 4 mm to 15 mm. In some examples, the unexpanded diameter can range from 3 mm to 6 mm.

In some examples, in the expanded configuration, the expansion element has any regular or irregular shaped (for example, radial and/or longitudinal) cross-section.

In some examples, in the expanded configuration, the expansion element has a curvilinear shaped radial and/or longitudinal cross-section.

In some examples, in the expanded configuration, the expansion element has a circular shaped radial and/or longitudinal cross-section.

In some examples, in the expanded configuration, the expansion element has a rectilinear shaped radial and/or longitudinal cross-section.

In some examples, in the expanded configuration, the expansion element has an elliptical shaped longitudinal cross-section.

In some examples, in the expanded configuration, the expansion element projects beyond an outer surface around an entire circumference of the dilator.

In some examples, in the expanded configuration, the expansion element projects beyond an outer surface around a portion of an entire circumference of the dilator.

In some examples, the expansion element is composed of a semi-compliant (high-strength) material (for example, HDPE or fluoropolymers) and/or include a low friction coating or hydrophilic polymer blend (for example, hydrophilic coating or hydrophilically lubricious blend). In some examples, the (outer layer) of the dilator is composed of nylon 12.

In some examples, the expansion element resists a burst pressure of at least about 5 atm.

In some examples, in the unexpanded configuration, the expansion element is folded against the dilator shaft, wherein an outer diameter of the of the expansion element in the unexpanded configuration is less than or equal to an outer diameter of the inflatable dilator.

In some examples, the dilator shaft includes a reduced diameter portion, wherein the unexpanded configuration, the expansion element is folded against the reduced diameter portion such that the outer diameter of the expansion element is not greater than an outer diameter of the dilator shaft (for example, outer surface of the dilator shaft).

In some examples, in the unexpanded configuration, the expansion element is folded against the dilator shaft in a propeller fold configuration (for example, including 5-8 folds).

In some examples, the reduced diameter portion includes a distally-tapered rear wall (for example, to provide space for the deflated expansion element to help with retrieval/removal from the patient's vasculature).

In some examples, the expansion element is covered by an elastomer cover.

In some examples, the elastomer cover restrains the expansion element in the unexpanded configuration and moves with the expansion element during expansion. In some examples, the elastomer cover has a low expansion force (for example, has Shore A durometer lower than 95A).

In some examples, the dilator shaft includes an elongated body portion adjacent a proximal end of the dilator shaft, and a tapered portion extending from a distal end of the dilator shaft toward the body portion.

In some examples, dilator/dilator shaft has a Shore D durometer ranging from 63D to 75D.

In some examples, the expansion element is provided on the body portion.

In some examples, the expansion element is provided on the body portion proximate the distal end of the dilator shaft (for example, at a location between the midpoint of the dilator shaft and the distal end of the dilator shaft).

In some examples, the dilator is received (for example, fully received) within the central lumen of the sheath, the expansion element is located beyond the distal end (for example, through and/or beyond the opening at the distal end of the sheath) of the sheath.

In some examples, in this position, the expansion element can be used to dilate the patient blood vessel, used to dilate a tight or hardened vessel, or disrupt/dislodge a calcific lesion, or other blockage/occlusion that may impede delivery of the medical device to the treatment location.

In some examples, the dilator can have a length ranging from 25 cm to 100 cm. In some examples, dilator is approximately 45 cm long. In some examples, the expansion element can be located 3 inches to 5 inches from the distal end of the dilator.

In some examples, the dilator further includes an inflation lumen in fluid communication with the inflation chamber for providing an inflation fluid thereto. In some examples, the inflation lumen is in fluid communication with an inflation port provided on the dilator hub.

In some examples, the dilator further includes in inner surface, outer surface, and a central lumen passing therethrough. In some examples, the central lumen is sized and configured to receive a guidewire. In some examples, the central lumen has a diameter of approximately 0.030 inches. In some examples, the central lumen is defined by the inner surface of the dilator.

In some examples, the inner surface is composed of a low friction material (for example, HDPE, fluoropolymers), a low friction coating, and/or a hydrophilic material or coating (for example, a hydrophilic polymer blend, hydrophilic coating, hydrophilically lubricious blend). In some examples, the dilator (for example, the outer layer of the dilator) is composed of nylon 12.

In some examples, the inflation lumen extends longitudinally within a wall thickness the dilator between the inner surface and the outer surface of the dilator.

In some examples, the inflation lumen has a regular or irregular shaped cross-section.

In some examples, the inflation lumen has a circular shaped cross-section.

In some examples, the inflation lumen has a crescent shaped cross-section.

In some examples, the inflation lumen has an annular shaped cross-section.

In some examples, dilator includes a dilator hub coupled to a proximal end of the dilator shaft, the dilator hub including an inflation port in fluid communication with the inflation lumen.

Another implementation of the present disclosure is directed to method of dilating a patient blood vessel. The method includes: providing a radially expandable sheath including a continuous inner layer defining a central lumen therethrough, the inner layer having at least one folded portion extending along a length of the inner layer; and advancing a dilator into the central lumen of the sheath with an inflatable expansion element provided on the dilator in an unexpanded configuration.

In some examples, the dilator includes an elongated dilator shaft with the expansion element including an inflation chamber (for example, a fluid-bearing chamber) provided thereon.

In some examples, where upon receipt of an inflation fluid within the inflation chamber, the expansion element is movable from the unexpanded in which the expansion element has a first diameter to an expanded configuration in which the expansion element has a second, larger diameter, and upon removal of the inflation fluid from the inflation chamber the expansion element returns to the unexpanded configuration.

In some examples, the method further includes: inserting the sheath at least partially into the blood vessel of the patient; advancing the dilator within the central lumen of the sheath and beyond the distal opening of the sheath until the expansion element is positioned at a treatment site within the patient's blood vessel; providing an inflation fluid to the inflation chamber thereby moving the expansion element from the unexpanded configuration to the expanded configuration and expanding the patient's blood vessel; removing the inflation fluid from the inflation chamber thereby moving the expansion element from the expanded configuration to the unexpanded configuration; and withdrawing the dilator from the central lumen of the sheath.

Another example of the present disclosure is directed to a method of delivering a medical device through a sheath. In some examples, the method includes: providing a radially expandable sheath including a continuous inner layer defining a central lumen therethrough, the inner layer having at least one folded portion extending along a length of the inner layer; and advancing a dilator into the central lumen of the sheath with an inflatable expansion element provided on the dilator in an unexpanded configuration.

In some examples, the dilator includes an elongated dilator shaft with the expansion element including an inflation chamber (for example, a fluid-bearing chamber) provided thereon, where upon receipt of an inflation fluid within the inflation chamber, the expansion element is movable from the unexpanded in which the expansion element has a first diameter to an expanded configuration in which the expansion element has a second, larger diameter, and upon removal of the inflation fluid from the inflation chamber the expansion element returns to the unexpanded configuration.

In some examples, the method further includes: inserting the sheath and coupled dilator at least partially into the blood vessel of the patient; advancing the dilator within the central lumen of the sheath and beyond the distal opening of the sheath until the expansion element is positioned at a treatment site within the patient's blood vessel; providing an inflation fluid to the inflation chamber thereby moving the expansion element from the unexpanded configuration to the expanded configuration and expanding the patient's blood vessel; removing the inflation fluid from the inflation chamber thereby moving the expansion element from the expanded configuration to the unexpanded configuration; withdrawing the dilator from the central lumen of the sheath; advancing a medical device through the central lumen of the sheath causing the sheath to locally expand from the unexpanded configuration to the expanded configuration at a location proximate the medical device in response to the outwardly directed radial force of the medical device exerted against the inner layer and locally contracting the sheath at least partially back to the unexpanded configuration as the medical device passes through the central lumen; and advancing the medical device beyond the distal opening of the sheath to the treatment site.

In some examples, the inner layer includes at least one folded portion, wherein locally expanding the central lumen of the sheath causes a length of the folded portion to at least partially unfold.

In some examples, sheath further includes: an outer layer provided over the inner layer, where the outer layer is discontinuous and includes an overlapping portion and an underlying portion, where when the sheath is in the unexpanded configuration, the overlapping portion overlaps the underlying portion with the folded portion of the inner layer disposed between the overlapping portion and the underlying portion, and a tubular strain relief layer provided over the inner layer and limits radial expansion of the sheath. In some examples, the strain relief layer extends at least partially over the outer layer.

In some examples, the medical device is a prosthetic device mounted in a radially crimped state on a delivery apparatus, and the act of advancing the prosthetic device through the central lumen of the sheath comprises advancing the delivery apparatus and the prosthetic device through central lumen of the sheath and into the vasculature of the patient.

In some examples, the prosthetic device comprises a prosthetic heart valve and the method further comprises implanting the prosthetic heart valve at a treatment site within the patient.

In some examples, the prosthetic heart valve is mounted on a balloon catheter of a delivery apparatus as the prosthetic heart valve is advanced through the sheath.

In some examples, the sheath is inserted into a femoral artery of the patient.

Various aspects of the examples described herein can be combined based on desired sheath system characteristics.

### BRIEF DESCRIPTION OF DRAWINGS

FIG. 1 is an elevation view of an expandable sheath along with an endovascular delivery apparatus for implanting a prosthetic implant.

FIG. 2 is an elevation view of an expandable sheath including an introducer locking hub, a sheath locking sleeve, and an introducer.

FIG. 3 is an elevation view of the expandable sheath of FIG. 2 along with an endovascular delivery apparatus for implanting a prosthetic implant.

FIG. 4 is an elevation view of an expandable sheath a sheath hub, an introducer locking hub, and a sheath locking sleeve of FIG. 2.

FIG. 5A is a cross-sectional view of the sheath hub, introducer locking hub, and sheath locking sleeve of FIG. 2.

FIG. 5B is a cross-sectional view of the introducer cap, the sheath hub, the introducer locking hub, the sheath locking sleeve of FIG. 2.

FIG. 6 is a cross-sectional view of the introducer cap, sheath hub, introducer locking hub, and sheath locking sleeve of FIG. 2.

FIG. 7 is a distal end view of the sheath locking sleeve of FIG. 2 and the proximal fluid seal of FIGS. 5A-B.

FIG. 8A is a first elevation view of the introducer locking hub of FIG. 2 coupled to an introducer.

FIG. 8B is a second (rotated) elevation view of the introducer locking hub of FIG. 2 coupled to the introducer.

FIG. 8C is a distal end view of the introducer locking hub of FIG. 2 coupled to the introducer.

FIG. 8D is a partial side view of the introducer locking hub of FIG. 2 coupled to the introducer.

FIG. 8E is a partial perspective view of the introducer locking hub of FIG. 2 coupled to the introducer.

FIG. 8F is a partial perspective view of the introducer locking hub of FIG. 2 coupled to the introducer.

FIG. 9A is a distal end view of the introducer locking hub of FIG. 2.

FIG. 9B is a first elevation view of the introducer locking hub of FIG. 2.

FIG. 9C is a proximal end view of the introducer locking hub of FIG. 2.

FIG. 9D is a first perspective view of the introducer locking hub of FIG. 2.

FIG. 9E is a second elevation view of the introducer locking hub of FIG. 2.

FIG. 9F is a second perspective view of the introducer locking hub of FIG. 2.

FIG. 10A is a distal end view of the sheath locking sleeve of FIG. 2.

FIG. 10B is a first elevation view of the sheath locking sleeve of FIG. 2.

FIG. 10C is a proximal end view of the sheath locking sleeve of FIG. 2.

FIG. 10D is a first perspective view of the sheath locking sleeve of FIG. 2.

FIG. 10E is a second elevation view of the sheath locking sleeve of FIG. 2.

FIG. 10F is a second perspective view of the sheath locking sleeve of FIG. 2.

FIG. 11 is a side elevation cross-sectional view of a portion of the expandable sheath of FIGS. 1 and 2.

FIG. 12 is a magnified view of a portion of the expandable sheath of FIGS. 1 and 2.

FIG. 13A is a magnified view of a portion of the expandable sheath of FIGS. 1 and 2 with the outer layer removed for purposes of illustration.

FIG. 13B is a magnified view of a portion of the braided layer of the sheath of FIGS. 1 and 2.

FIG. 14 is a magnified view of a portion of the expandable sheath of FIGS. 1 and 2 illustrating expansion of the sheath as a prosthetic device is advanced through the sheath.

FIG. 15 is a side view of the expandable sheath of FIGS. 1 and 2.

FIG. 16 is a magnified cross-sectional section view of the sheath of FIG. 15 along section line 16-16.

FIG. 17 is cross-sectional view of the unexpanded sheath of FIG. 16 along section line 17-17.

FIG. 18 is cross-sectional view of the unexpanded sheath of FIG. 15 along section line 18-18.

FIG. 19 is cross-sectional view of the unexpanded sheath of FIG. 15 along section line 19-19.

FIG. 20 is cross-sectional view of the expanded sheath of FIG. 15 along section line 19-19.

FIG. 21 is a side view of the expandable sheath of FIGS. 1 and 2.

FIG. 22 is a cross-sectional view of the unexpanded sheath of FIG. 21 along section line 22-22.

FIG. 23 is a cross-sectional view of the expanded sheath of FIG. 21 along section line 22-22.

FIG. 24 is a side view of the expandable sheath of FIGS. 1 and 2 and an example dilator.

FIG. 25 is a side view of the dilator of FIG. 25 in an unexpanded configuration.

FIG. 26 is a side view of the dilator of FIG. 25 in an expanded configuration.

FIG. 27 is a magnified view of the longitudinal cross-section of the distal end of the dilator of FIG. 25 in an unexpanded configuration.

FIG. 28 is a magnified view of the longitudinal cross-section of the distal end of the dilator of FIG. 25 in an expanded configuration.

FIG. 29 is a magnified view of the longitudinal cross-section of the distal end of the dilator of FIG. 25 in an expanded configuration according to another implementation.

FIG. 30 is a cross-sectional view of the dilator of FIG. 24 taken along section line 30-30.

FIG. 31 is a cross-sectional view of the dilator of FIG. 24 taken along section line 30-30 according to another implementation.

FIG. 32 is a cross-sectional view of the dilator of FIG. 24 taken along section line 30-30 according to another implementation.

### DETAILED DESCRIPTION

The following description of some examples of the inventive concepts should not be used to limit the scope of the claims. Other examples, features, aspects, implementations, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect or example of the present disclosure are to be understood to be applicable to any other aspect or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing examples. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Disclosed examples of an expandable sheath can minimize trauma to the vessel by allowing for temporary expansion of a portion of the introducer sheath to accommodate the delivery apparatus, followed by a return to the original diameter once the device passes through. Disclosed examples of the introducer sheath prevent the introducer/dilator from separating from the sheath during insertion by locking of the proximal hub of the introducer/dilator to the proximal hub of the sheath. Fixing the introducer/dilator and the sheath prevents the introducer/dilator from moving backward during insertion, thereby maintaining a snug fit and smooth transition between the introducer/dilator and the distal end of the sheath. Furthermore, present examples can reduce the length of time a procedure takes, as well as reduce the risk of a longitudinal or radial vessel tear, or plaque dislodgement because only one sheath is required, rather than several different sizes of sheaths. Examples of the present expandable sheath can avoid the need for multiple insertions for the dilation of the vessel.

Disclosed herein are elongate introducer sheaths that are particularly suitable for delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, and also in U.S. Pat. No. 9,393,110, both of which are hereby incorporated by reference. The expandable introducer sheaths disclosed herein may also be used to deliver other types of implantable medical device, such as self-expanding and mechanically expanding implantable heart valves, stents or filters. Beyond transcatheter heart valves, the introducer sheath system can be useful for other types of minimally invasive surgery, such as any surgery requiring introduction of an apparatus into a subject's vessel. For example, the introducer sheath system can be used to introduce other types of delivery apparatus for placing various types of intraluminal devices (for example, stents, stented grafts, balloon catheters for angioplasty procedures, etc.) into many types of vascular and non-vascular body lumens (for example, veins, arteries, esophagus, ducts of the biliary tree, intestine, urethra, fallopian tube, other endocrine or exocrine ducts, etc.). The term “implantable” as used herein is broadly defined to mean anything—prosthetic or not—that is delivered to a site within a body. A diagnostic device, for example, may be an implantable.

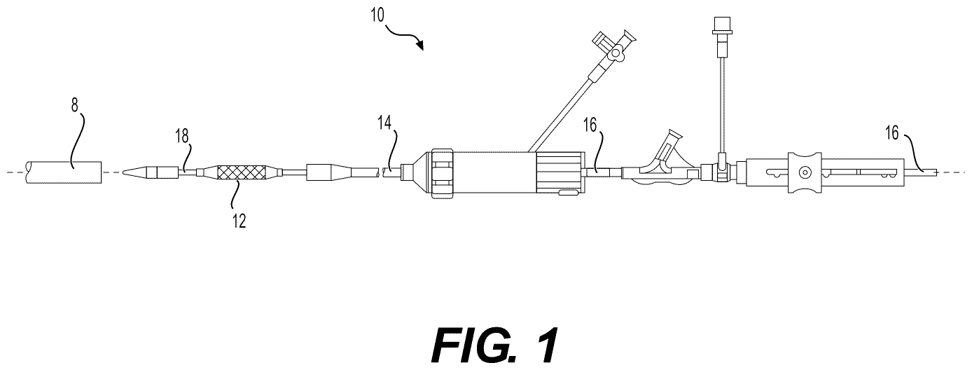

FIG. 1 illustrates an exemplary sheath 8 in use with a representative delivery apparatus 10, for delivering an implant 12 to a patient. As provided herein, the implant 12 can be another type of implantable medical devices (for example, a tissue heart valve). The delivery apparatus 10 can include a steerable guide catheter 14 (also referred to as a flex catheter) and a balloon catheter 16 extending through the guide catheter 14, and a nose catheter 15 extending through the balloon catheter 16. The guide catheter 14, balloon catheter 16, and nose catheter 15 in the illustrated example are adapted to slide longitudinally relative to each other to facilitate delivery and positioning of the implant 12 at an implantation site in a patient's body as described in detail herein. It is contemplated that the sheath 8 can be used with any type of elongated delivery apparatus used for implanting balloon-expandable prosthetic valves, self-expanding prosthetic valves, and other prosthetic devices.

As described in more detail herein, in general, the sheath 8 comprises an elongate expandable tube that, in use, is inserted into a vessel (for example, transfemoral vessel, femoral artery, iliac artery) by passing through the skin of patient, such that the distal end of the sheath 8 is inserted into the vessel. Sheath 8 includes a hemostasis valve and/or sealing features at the proximal end of the sheath 8, for example, in the sheath hub 20, that provide hemostasis and prevents blood leakage from the patient through the sheath 8. The sheath 8, including an introducer 6, is advanced into the patient's vasculature. Once positioned the introducer 6 is removed and the delivery apparatus 10 is inserted into/through the sheath 8, and the prosthetic device (for example, implant 12) then be delivered and implanted within patient.

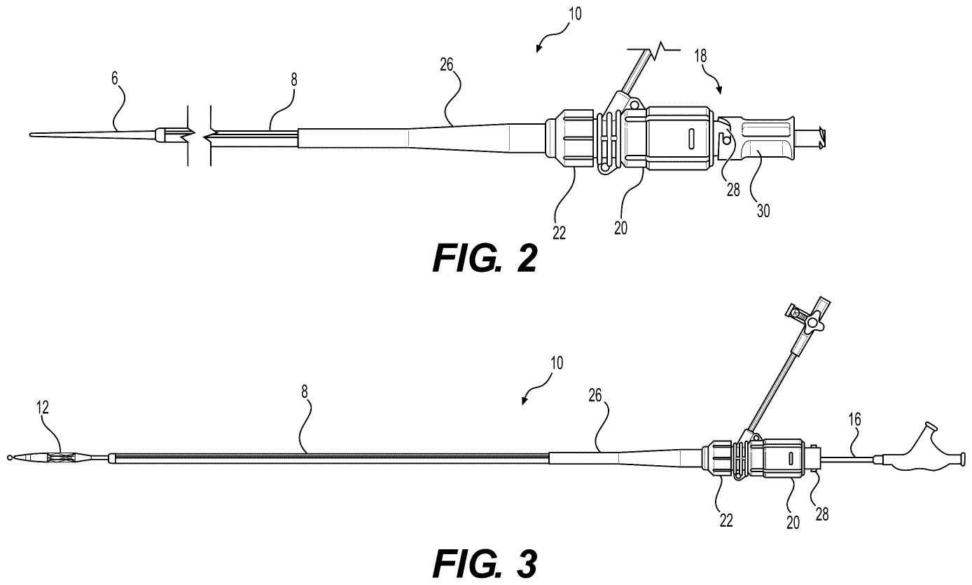

FIGS. 2 and 3, the introducer device/sheath assembly includes a sheath hub 20 at a proximal end of the device and an expandable sheath 8 extending distally from the sheath hub 20. The sheath 8 is coupled to the sheath hub 20 which in turn is removably coupled to a sheath locking system 18. The sheath locking system 18 allows the introducer 6, or other device desired to be removably couped (axially and rotatably) to the sheath 8.

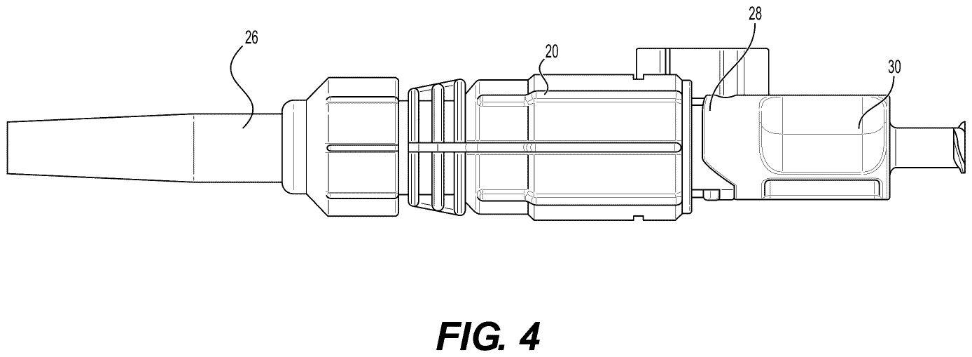

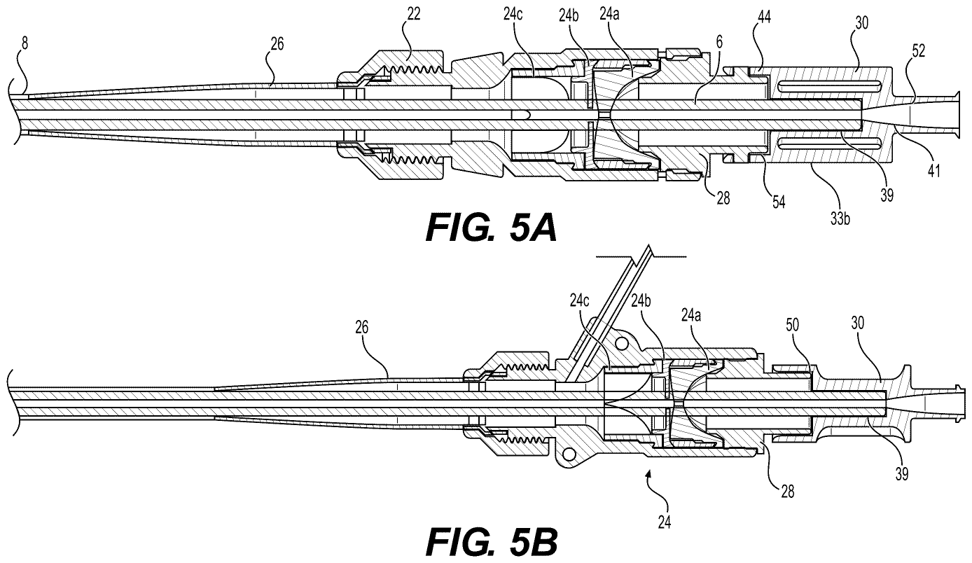

As illustrated in FIGS. 2-6, the sheath hub 20 can function as a handle for the device. Sheath hub 20 also provides a housing for necessary seal assemblies and an access point for a secondary lumen (for example, fluid lumen) in fluid communication with the central lumen of the sheath hub 20. The seal assembly 24, as described herein and as shown in FIGS. 5A and 5B, is included in the sheath hub 20. In some examples, the seal assembly 24 includes a proximal seal 24a, an intermediate seal 24b, and a distal seal 24c. When assembled, the introducer 6 passes through the seal assembly and extends distal of the sheath 8. The proximal seal 24a, the intermediate seal 24b, and the distal seal 24c are each formed to prevent unwanted fluid from advancing in the proximal direction through the sheath hub 20 and proximal of the seal assembly 24. They are each openable and closable to provide pressure variation to affect the desired fluid flow from a physician or technician.

In some examples, the distal end of the sheath hub 20 includes threads 21 for coupling to a threaded sheath hub cap 22. The sheath 8 is provided between the sheath hub 20 and the sheath hub cap 22 such that coupling the sheath hub cap 22 to the sheath hub 20 fixes the sheath 8 to the sheath hub 20. The sheath hub cap 22 is a cylindrical cap having a cap body having a proximal end and a distal end and defining a central lumen extending longitudinally between the proximal end and the distal end. The sheath hub cap 22 has a larger diameter at its proximal end than at its distal end.

In some examples, the sheath hub 20 further has receiving slots 48 for coupling the sheath locking system 18, particularly the locking sleeve 28, to the sheath hub 20. The receiving slots 48 are openings which extend around a portion of the diameter of the sheath hub 20 and are sized and configured to accept the interference diameters 66 of the locking sleeve 28. Coupling between the receiving slots 48 and the interference diameters 66 axially and rotationally fixes the locking sleeve 28 and the sheath hub 20 relative to each other.

FIG. 2 illustrates the sheath 8 of FIG. 1 including a sheath locking system 18 which prevents axial and rotational translation of the introducer 6 with respect to the sheath 8. Example locking systems are disclosed in PCT/US2021/050006, entitled “Expandable Sheath Including Reverse Bayonet Locking Hub,” the disclosure of which is incorporated herein by reference. It is contemplated that the locking system disclosed herein can also be used to couple the sheath 8/sheath hub 20 with other delivery apparatus components, catheters, dilators, etc. including the same mating features.

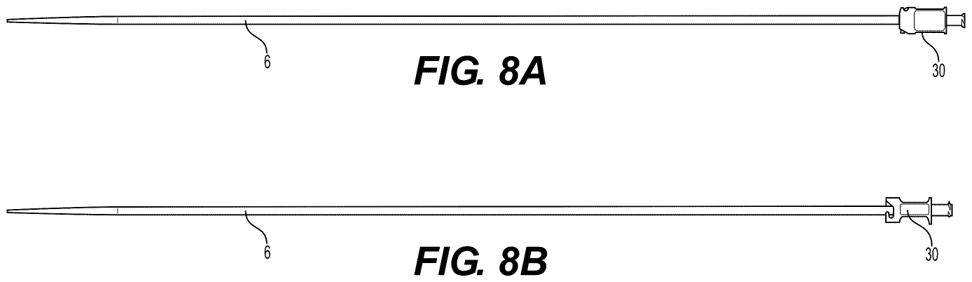

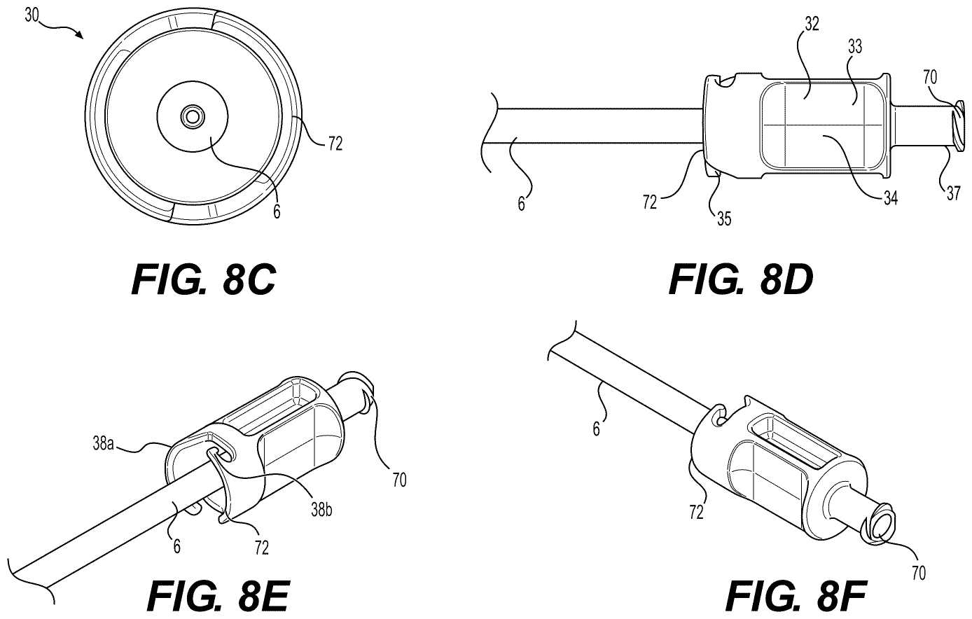

The sheath locking system 18 keeps the introducer 6 fixed with respect to the sheath 8 during insertion without requiring a physician or technician to hold the introducer 6 and the sheath 8 in place at the distal end. As illustrated in FIGS. 8A-8B, the sheath locking system 18 includes a locking sleeve 28 and an introducer locking hub 30 (including corresponding introducer 6). The locking sleeve 28 is coupled to the sheath 8 via the sheath hub 20. The locking sleeve 28 engages the introducer locking hub 30 and is moveable between a locked and unlocked position, thereby fixing the position of the introducer 6 and the sheath 8 and preventing movement therebetween, particularly during insertion into the patient. As will be described in more detail herein, the sheath locking system 18 keeps the introducer 6 from separating from the sheath 8 and prevents gaps from forming that can cause patient abrasions and unintended fluid flow between the introducer 6 and the sheath 8 during insertion.

FIGS. 2, 5A-5B, and 6, and illustrate the locking sleeve 28 coupled to the introducer locking hub 30 and the sheath hub 20. As will be described in more detail herein, in some examples, the locking sleeve 28 includes a guide 31 that engages a locking channel 38 provided on the introducer locking hub 30. The guide 31 moves within the locking channel 38 between an unlocked position, where the locking sleeve 28 is rotationally and axially movable with respect to the introducer locking hub 30, and a locked position (FIG. 2), where the locking sleeve 28 is axially fixed with respect to the introducer locking hub 30.

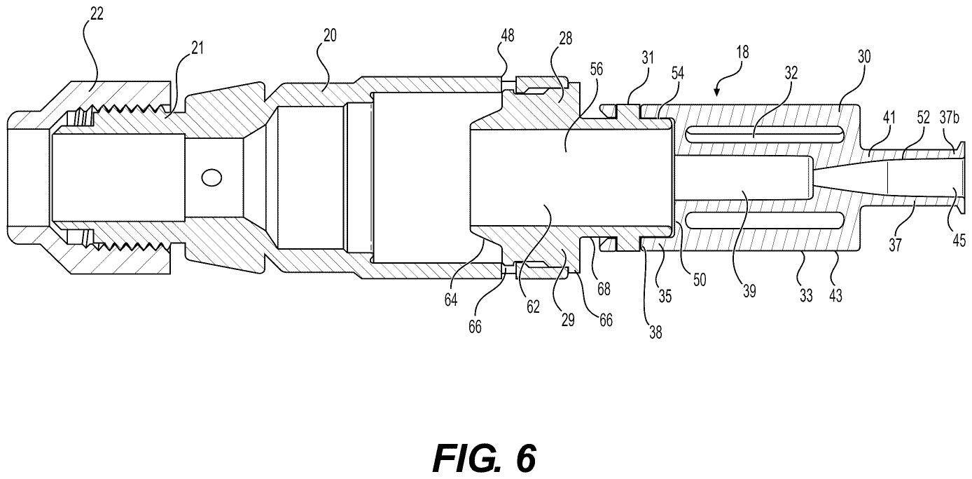

The locking sleeve 28 is illustrated, for example, in FIGS. 10A-10F. In some examples, the locking sleeve 28 includes an elongated sleeve body 29 with a central lumen 56 extending longitudinally between the proximal end 58 and distal end 60 of the sleeve body 29. As provided in FIG. 6, the central lumen 56 defines a generally cylindrical inner surface 62 of the sheath locking sleeve 28. The central lumen 56 has a diameter of at least 0.3 inches. In some examples, the diameter ranges between 0.3 inches and 0.6 inches. Preferably, the diameter is about 0.40 inches. The distal end 60 of the sleeve body 29 also has a frustoconical outer surface 64 that tapers about the distal end 60 to help with positioning the locking sleeve 28 within the sheath hub 20 and abutting the seal assembly 24 (FIGS. 5B and 5B). The locking sleeve 28 also has a plurality of interference diameters 66 that extend radially from the outer surface of the sleeve body 29 around (all or a portion of) the circumference of the locking sleeve 28. As illustrated in FIGS. 5A and 6, the distal interference diameters 66 are sized and configured to engage corresponding recesses and/or slots 48 provided in the sheath hub 20 for securing the locking sleeve 28 to the sheath hub 20, and the distal interference diameter 66 seat against the proximal end of the sheath hub 20.

The locking sleeve 28 includes a guide 31 projecting from the outer surface 68 of the locking sleeve 28. The guide 31 engages a corresponding shaped locking channel 38 in the introducer locking hub 30. The guide 31 extends radially from the outer surface 68 and at least partially around the circumference of the outer surface 68. As provided in FIG. 6, the top surface of the guide 31 does not extend beyond the outer surface of the introducer locking hub 30 when the locking sleeve 28 and the introducer locking hub 30 are coupled. For example, the height of the guide 31 corresponds to the wall thickness of the introducer locking hub 30 proximate the guide when the locking sleeve 28 and the introducer locking hub 30 are coupled. In some examples, the top surface of the guide 31 is recessed with respect to the outer surface of the introducer locking hub 30. That is, the height of the guide 31 is less than the wall thickness of the introducer locking hub 30. In some examples, the height of the guide 31 is greater than a wall thickness of the introducer locking hub 30 such that the top surface of the guide 31 extends beyond the outer surface of the introducer locking hub 30 when the locking sleeve 28 and the introducer locking hub 30 are coupled. In some examples, the height/axial length of the guide 31 is between about 0.050 inches and about 0.10 inches. In some examples that height/axial length of the guide 31 is about 0.075 inches.

As illustrated in FIGS. 10D-10F, the guide 31 is a cylindrically shaped projection. However, it is contemplated that the guide 31 may have any other regular or irregular shape that would facilitate movement of the guide 31 within the locking channel 38 of the introducer locking hub 30. For example, the guide 31 may have an elongated hexagon shape. The guide 31 can have a diameter/width ranging from about 0.05 inches to about 0.20 inches. Preferably the guide 31 has a diameter/width of about 0.100 inches.

In general, the locking sleeve 28 can comprise polycarbonate, but in some examples, the locking sleeve 28 can comprise rigid plastic, or any other material suitable for providing a strong locking connector for an introducer 6 (metal, composite, etc.).

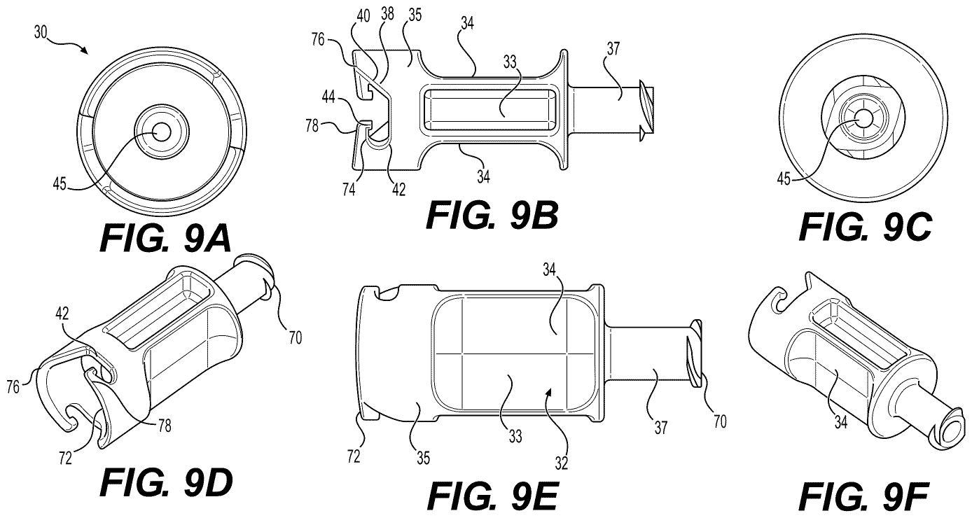

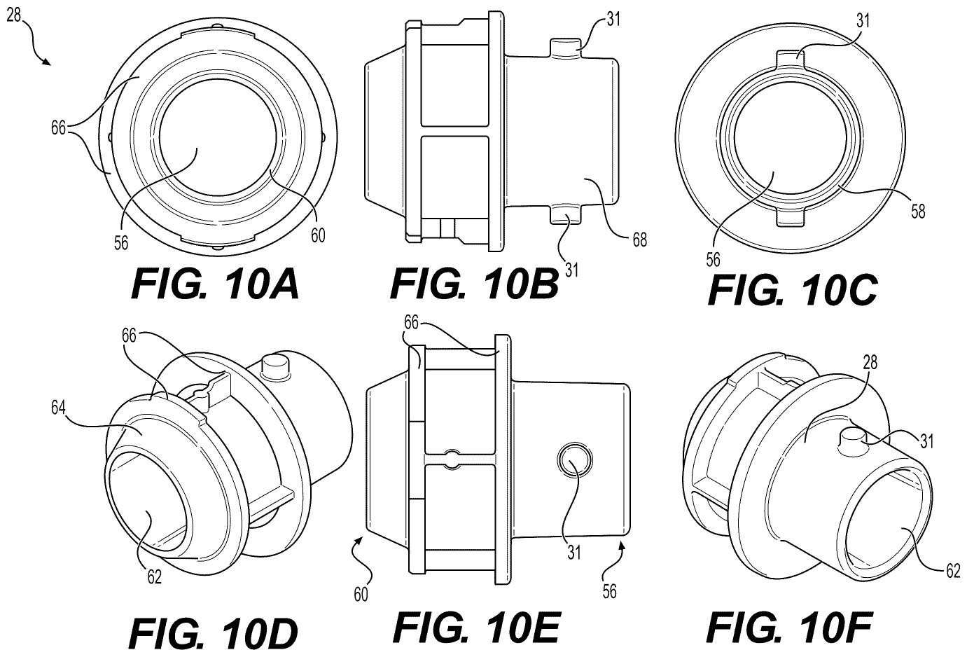

FIGS. 2-6 illustrate the introducer locking hub 30 coupled to the locking sleeve 28. FIGS. 8A-8F show the introducer locking hub 30 coupled to the introducer 6. FIGS. 9A-9F provide multiple view of the introducer locking hub 30. As described herein, the introducer 6 is fixedly coupled to the introducer locking hub 30. The introducer locking hub 30 couples with the locking sleeve 28 to fix the position the introducer 6 (axially and rotationally) with respect to the locking sleeve 28/sheath 8. Each of the introducer 6 and introducer locking hub 30 are described in more detail herein.

FIGS. 8A-8F illustrate the introducer locking hub 30 with the introducer 6 coupled thereto. Example introducer sheaths are described, for example in U.S. Pat. Nos. 8,690,936 and 8,790,387, the disclosures of which are incorporated herein by reference. As provided in the cross-sectional views of FIGS. 5A and 5B, the introducer 6 is coupled to the introducer locking hub 30 and extends beyond the distal end of the introducer locking hub 30 body and into the sheath 8. When coupled to the sheath hub 20, the introducer 6 extends through the central lumen 56 of the locking sleeve 28, the sheath hub 20 and the central lumen of the sheath 8. As will be descried herein, the sheath 8 generally comprises a radially expandable tubular structure. Passage of the introducer 6 through the sheath 8 and into a patient's vasculature causes the blood vessel to radially expand to about the diameter of the sheath 8. That is, the diameter of the central lumen of the sheath 8 is generally abuts the outer diameter of the introducer 6 such that the introducer 6 provides a mechanism to expand a patient's vessel to accept the sheath.

As provided in FIGS. 8A-8F, the introducer 6 is formed as an elongate body with a central lumen extending therethrough. As shown in FIGS. 5A and 5B, the central lumen of the introducer is aligned with the central lumens of the introducer locking hub 30, the sheath hub 20 and the sheath 8. In some examples, the introducer 6 is received within a recessed opening 39 provided on an interior surface of the introducer locking hub 30, the recessed opening 39 axially aligned with the central lumen 45 of the introducer locking hub 30. The introducer 6 is coupled to the introducer locking hub 30 at the recessed opening 39. In an example system, the introducer 6 has a diameter corresponding to, or less than, the diameter of the recessed opening 39. In some examples, the introducer 6 is fixedly coupled to the introducer locking hub 30 at the recessed opening 39. For example, the introducer 6 is coupled to the recessed opening 39 of the introducer locking hub 30 by at least one of a press fit, an interference fit, a snap fit, a mechanical fastener, a chemical fastener (for example, an adhesive), a weld, a thermal process, and/or any other suitable coupling process known in the art.

As described herein, the introducer 6 has a central lumen that aligns with the central lumen 45 of the introducer locking hub 30. This joined lumen allows for the passage of surgical equipment and/or medical devices to the treatment site (for example, a guide wire). In an example system, and as provided in FIGS. 5A and 5B, the central lumen of the introducer 6 has a diameter corresponding to at least a portion of the diameter of the central lumen 45 of the introducer locking hub 30. In general, the corresponding diameter portion is adjacent the distal end of the central lumen 45. In some examples, the diameter of the central lumen 45 at the distal end of the introducer locking hub 30 is slightly larger than the diameter of the central lumen passing through the introducer 6. The central lumen 45 can also define a decreasing tapered portion 41 between the proximal end and the distal end of the introducer locking hub 30 (see FIG. 6). The corresponding diameter portion and decreasing tapered portion 41 allows for smooth transition and delivery of surgical equipment and/or medical device (such as implant 12) through the introducer locking hub 30 and into the central lumen of the introducer 6.

As illustrated in FIGS. 9A-9F, In some examples, the introducer locking hub 30 includes an introducer locking hub body 32 having a proximal end 70 and a distal end 72 and defining a central lumen 45 extending therethrough. The introducer locking hub body 32 has a first (middle) portion 33, a second (distal) portion 35 which extends distally from the first portion 33 and a third (proximal) portion 37 which extends proximally from the first portion 33. The first portion 33 includes the cylindrically-shaped recessed opening 39 for receiving and retaining the introducer 6 and an outer surface 43. In some examples, the recessed opening 39 has a diameter ranging between 0.15 inches and about 0.25 inches. In some examples, the recessed opening 39 has a diameter ranging between 0.17 inches and about 0.20 inches. In some examples, the recessed opening has a diameter of about 0.194 inches.

The third (proximal) portion 37 of the introducer locking hub 30 includes the decreasing tapered portion 41 of the central lumen 45. The decreasing tapered portion 41 defining a frustoconical shape with decreasing taper/diameter from the proximal to the distal end of the sheath 8. It is contemplated that the tapered portion 41 has a minimum diameter of about 0.007 inches and a maximum diameter of about 0.194 inches.

As illustrated in FIGS. 5A and B, when coupled, the central lumen 56 of the locking sleeve 28 is aligned with the central lumen 45 of the introducer locking hub 30. In some examples, the central lumen 56 of the locking sleeve 28 is coaxial with the central lumen 45 of the introducer locking hub 30. When coupled, the proximal end of the locking sleeve 28 is received within the central lumen 45 of the introducer locking hub 30. The proximal end surface of the locking sleeve 28 is adjacent a shoulder 50 provided on an inner surface of the central lumen 45 of the introducer locking hub 30. As illustrated in FIGS. 5A and 5B, the central lumen 45 of the introducer locking hub 30 includes a first portion 52 having a first diameter adjacent the proximal end of the introducer locking hub 30, and a second portion 54 having a second, larger, diameter adjacent the distal end of the introducer locking hub 30. The recessed opening 39 can be considered either a component of the first portion 52 of the central lumen 45, or a separate component of the central lumen 45 located between the first (proximal) portion 52 and the second (distal) portion 54. When the locking sleeve 28 and introducer locking hub 30 are coupled, at least a portion of the sleeve body 29 of the locking sleeve 28 is received within the second portion 54 (larger portion) of the central lumen 45 of the introducer locking hub 30. The central lumen 56 of the locking sleeve 28 is aligned with the central lumen 45 of the introducer locking hub 30 such that they are co-axial and form a smooth inner surface along the combined central lumens of the introducer locking hub 30 and the locking sleeve 28.

As described herein, the locking sleeve 28 couples to the introducer locking hub 30 via engagement between the guide 31 on the locking sleeve 28 and the locking channel 38 provided in the introducer locking hub 30. As provided in FIGS. 9A-9F, the introducer locking hub 30 includes two locking channels 38. However, it is contemplated that the introducer locking hub 30 can include one locking channel 38 or more than two locking channels 38. The locking channel 38 can be is formed a recess or groove in a surface of the introducer locking hub 30, as a slotted opening, a clip, or as any other feature capable of receiving and securing the guide 31 projecting from the outer surface of the locking sleeve 28 with the introducer locking hub 30. Illustrated in FIG. 9B, the locking channels 38 provide an interface to secure the locking sleeve 28 to the introducer locking hub 30 and ensure a fixed axial position between the introducer 6 and the sheath 8.

The locking channel 38 is formed on the distal end of the introducer locking hub 30. The locking channel 38 includes an opening on the distal end surface that leads to an angled guide portion 40 that transitions to a locking portion 42. The guide portion 40 is configured to direct the guide 31 of the locking sleeve 28 in an axial and circumferential direction along the side wall of the guide portion 40 towards the locking portion 42 upon rotation of the introducer locking hub 30 and/or the locking sleeve 28. The locking portion 42 is configured to securely engage the guide 31, fixing the axial position of the introducer locking hub 30 with respect to the locking sleeve 28. As illustrated in FIG. 9B, the guide portion 40 of the locking channel 38 extends from the distal end of the introducer locking hub 30 axially towards the proximal end of the introducer locking hub 30 and circumferentially around the introducer locking hub 30. For example, the guide portion 40 of the locking channel 38 can be described as extending helically around/along a length of the introducer locking hub 30 or on an angle from the distal end of the introducer locking hub 30.

As illustrated in FIGS. 9B and 9D, the locking portion 42 of the locking channel 38 extends at an angle from the end of the guide portion 40. As provided in FIG. 9B, the angle between the centerline of the guide portion 40 and the centerline of the locking portion 42 is greater than 90-degrees. In some examples, the angle between the centerline of the guide portion 40 and the centerline of the locking portion 42 is about 120-degrees. In an example system, the locking portion 42 extends around a portion of the circumference of the introducer locking hub 30. The locking portion 42 can extend parallel to the distal end of the introducer locking hub 30. In an example system, the length of the guide portion 40 (measured along its centerline) is greater than a length of the locking portion 42 (measured along its centerline). In some examples, the length of the guide portion 40 equals or is less than a length of the locking portion 42.

The locking portion 42 can include a catch 44 for securing the guide 31 within the locking portion 42 of the locking channel 38 and forming a partial barrier for the guide 31 within the locking portion 42. As illustrated in FIG. 9B, the catch 44 includes a projection that extends from a side wall 74 of the locking portion 42 and releasably secures the guide 31 within the locking channel 38. The catch 44 extends from the side wall 42a of the locking portion 42 in a proximal direction towards the center line of the locking portion 42 and has a height sufficient to retain the guide 31 between the catch 44 and the end of the locking portion 42.

In some examples, the distal end surface 72 of the introducer locking hub 30 can include features for biasing the guide 31 towards and into the locking channel 38. For example, the distal end of the introducer locking hub 30 can include a tapered surface angled toward an opening of the locking channel 38. As illustrated in FIG. 9B, the distal end 72 of the introducer locking hub 30 includes a first tapered surface 76 (angled towards a leading edge of the opening of the locking channel 38 and a second tapered surface 78 angled towards the trailing edge of the opening of the locking channel 38.

In use, engagement between the guide 31 and the guide portion 40 of the locking channel 38 is configured to bias the locking sleeve 28 in a proximal axial direction toward the proximal end 70 of the introducer locking hub 30 (towards a locked position) when the locking sleeve 28 is rotated in a first axial direction. In this direction the guide 31 advances toward the locking portion 42 of the locking channel 38 into the locked position. Alternatively, engagement between the guide 31 and the locking portion 42 of the locking channel 38 is configured to bias the locking sleeve 28 in a distal axial direction toward the distal end of the introducer locking hub 30 (towards an unlocked position) when the locking sleeve 28 is rotated in a second (opposite) axial direction. In the second direction, the guide 31 advances away from the locking portion 42 of the locking channel 38, to the unlocked position. When the guide 31 is in the locked position and retained with by locking portion 42 by catch 44, rotation in the second direction causes the guide 31 to bias against the catch 44 overcoming the oppositional forces of the catch 44, and moving the guide 31 from the locked to the unlocked position.

As illustrated in FIGS. 8A-9F, In some examples, the outer surface of the introducer locking hub body 32 includes gripping features and/or surfaces for a physician or technician to use when manipulating the introducer locking hub 30. As provided in FIG. 9B, the introducer locking hub body 32 can include a two recessed gripping surfaces 34 on opposite sides of the longitudinal axis of the introducer locking hub 30. When the introducer locking hub 30 is viewed from the side, the gripping surfaces 34 define a dog-bone/barbell shape to the hub body 32, i.e., a shape having a smaller diameter/width center portion and larger diameter/width end portions. In an example system, the gripping surfaces 34 are provided along at least 40% of the length of the introducer locking hub body 32. In some examples, the gripping surfaces 34 are provided along at least 50% of the length of the introducer locking hub body 32.

In general, the introducer locking hub 30 can comprise polycarbonate, but in some examples the introducer locking hub 30 can comprise rigid plastic, or any other material suitable for providing a locking mechanism for an introducer 6 (metal, composite, etc.).

As described herein, the introducer device/sheath assembly includes an expandable sheath 8 extending distally from the sheath hub 20. The expandable sheath 8 has a central lumen to guide passage of the delivery apparatus 10 for the medical device/prosthetic heart valve (implant 12). In some examples, the introducer device/sheath assembly need not include the sheath hub 20. For example, the sheath 8 can be an integral part of a component of the sheath assembly, such as the guide catheter.

As described herein, the expandable sheath 8 can comprise a highly elastomeric materials that allows for the dilating of the vessel to be performed by the passing prosthetic device (implant 12). Example expandable introducer sheaths 8 are disclosed in U.S. Pat. No. 8,690,936, entitled “Expandable Sheath for Introducing an Endovascular Delivery Device into a Body,” U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. Pat. No. 10,792,471, entitled “Expandable Sheath,” U.S. patent application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. Pat. No. 11,273,062, entitled “Expandable Sheath,” Application No. PCT/US2021/019514, entitled “Expandable sheath for introducing an endovascular delivery device in to a body,” Application No. PCT/US2021/031227, entitled “Expandable sheath for introducing an endovascular delivery device into a body,” Application No. PCT/US2021/031275, entitled “Expandable sheath for introducing an endovascular delivery device into a body,” U.S. application Ser. No. 17/113,268, entitled “Expandable Sheath and Method of Using the Same,” Application No. PCT/US2021/058247, entitled “Self-Expanding, Two Component Sheath,” Application No. PCT/US2022/012785, entitled “Expandable Sheath,” U.S. Pat. No. 11,051,939, entitled “Active Introducer Sheath System,” Application No. PCT/US2022/012684, entitled “Introducer with Sheath Tip Expander,” U.S. application Ser. No. 17/078,556, entitled “Advanced Sheath Patterns,” Application No. PCT/US2021/025038, entitled “Low temperature hydrophilic adhesive for use in expandable sheath for introducing an endovascular delivery device into a body,” Application No. PCT/US2021/050006, entitled “Expandable Sheath Including Reversable Bayonet Locking Hub,” U.S. Provisional Application No. 63/280,251, entitled “Expandable Sheath Gasket to Provide Hemostasis,” the disclosures of which are herein incorporated by reference.

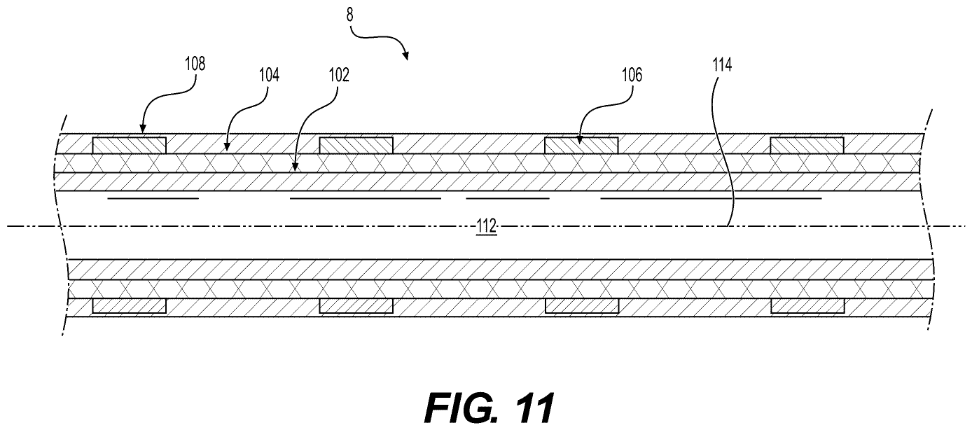

In some examples, the expandable sheath 8 can comprise a plurality of coaxial layers extending along at least a portion of the length of the sheath 8. The structure of the coaxial layers is described in more detail herein with respect to FIGS. 11-23. Example expandable sheaths including coaxial layers are described, for example, in U.S. patent application Ser. No. 16/378,417, entitled “Expandable Sheath,” and U.S. patent application Ser. No. 17/716,882, entitled “Expandable Sheath,” the disclosures of which are herein incorporated by reference.

Various examples of the coaxial layered structure of the sheath 8 are described herein. For example, in reference to the example sheath 8 illustrated in FIGS. 11-14, the expandable sheath 8 can include a number of layers including an inner layer 102 (also referred to as an inner layer), a second layer 104 disposed around and radially outward of the inner layer 102, a third layer 106 disposed around and radially outward of the second layer 104, and a fourth outer layer 108 (also referred to as an outer layer) disposed around and radially outward of the third layer 106. In the illustrated configuration, the inner layer 102 can define the lumen 112 of the sheath 8 extending along a central axis 114 through which the delivery apparatus 10 travels into the patient's vessel in order to deliver, remove, repair, and/or replace a prosthetic device (for example, implant 12), moving in a direction along the longitudinal axis of the sheath 8.

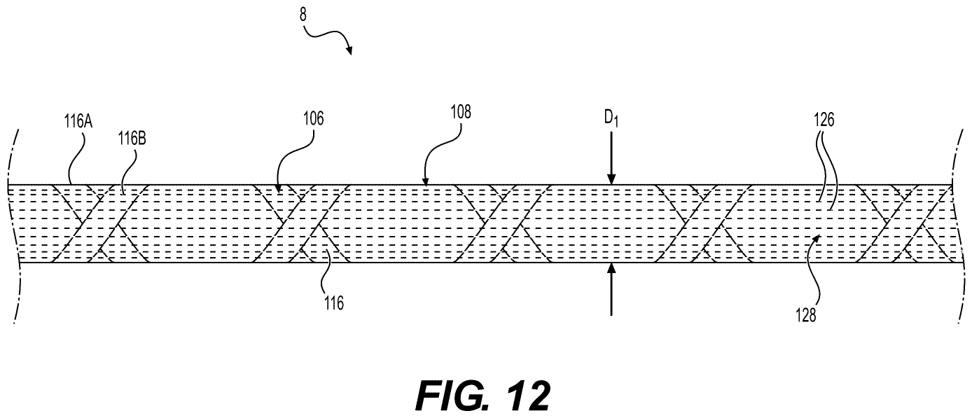

Referring to FIG. 12, when the sheath 8 is in an unexpanded state, various layers of the sheath 8, for example, the inner layer 102 and/or the outer layer 108, can form longitudinally-extending folds or creases such that the surface of the sheath 8 comprises a plurality of folds or ridges 126. The ridges 126 can be circumferentially spaced apart from each other by longitudinally-extending valleys 128. When the sheath 8 expands beyond its natural diameter D₁, the ridges 126 and the valleys 128 can level out or be taken up as the surface radially expands and the circumference increases, as further described herein. When the sheath 8 collapses back to its natural diameter, the ridges 126 and valleys 128 can reform.

In some examples, the inner layer 102 and/or the outer layer 108 can comprise a relatively thin layer of polymeric material. For example, the thickness of the inner layer 102 can be from 0.01 mm to 0.5 mm, 0.02 mm to 0.4 mm, or 0.03 mm to 0.25 mm. In some examples, the thickness of the outer layer 108 can be from 0.01 mm to 0.5 mm, 0.02 mm to 0.4 mm, or 0.03 mm to 0.25 mm.

In some examples, the inner layer 102 and/or the outer layer 108 can comprise a lubricious, low-friction, and/or relatively non-elastic material. For example, the inner layer 102 and/or the outer layer 108 can comprise a polymeric material having a modulus of elasticity of 400 Mpa or greater. Exemplary materials can include ultra-high-molecular-weight polyethylene (UHMWPE) (for example, Dyneema®), high-molecular-weight polyethylene (HMWPE), or polyether ether ketone (PEEK). With regard to the inner layer 102 in particular, such low coefficient of friction materials can facilitate passage of the prosthetic device (implant 12) through the lumen 112. Other suitable materials for the inner layer 102 and outer layer 108 can include polyimide, polytetrafluoroethylene (PTFE), expanded polytetrafluoroethylene (ePTFE), ethylene tetrafluoroethylene (ETFE), nylon, polyethylene, polyamide, polyether block amide (for example, Pebax), and/or combinations of any of the above. In some examples, the sheath 8 can include a lubricious liner on the inner surface of the inner layer 102. Examples of suitable lubricious liners include materials that can further reduce the coefficient of friction of the inner layer 102, such as PTFE, polyethylene, polyvinylidene fluoride, and combinations thereof. Suitable materials for a lubricious liner also include other materials desirably having a coefficient of friction of 0.1 or less.

Additionally, some examples of the sheath 8 can include an exterior hydrophilic coating on the outer surface of the outer layer 108. Such a hydrophilic coating can facilitate insertion of the sheath 8 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (for example, PTFE, polyethylene, polyvinylidene fluoride), are also suitable for use with the sheath 8. Such hydrophilic coatings may also be included on the inner surface of the inner layer 102 to reduce friction between the sheath 8 and the delivery apparatus 10, thereby facilitating use and improving safety. In some examples, a hydrophobic coating, such as Perylene, may be used on the outer surface of the outer layer 108 or the inner surface of the inner layer 102 in order to reduce friction.

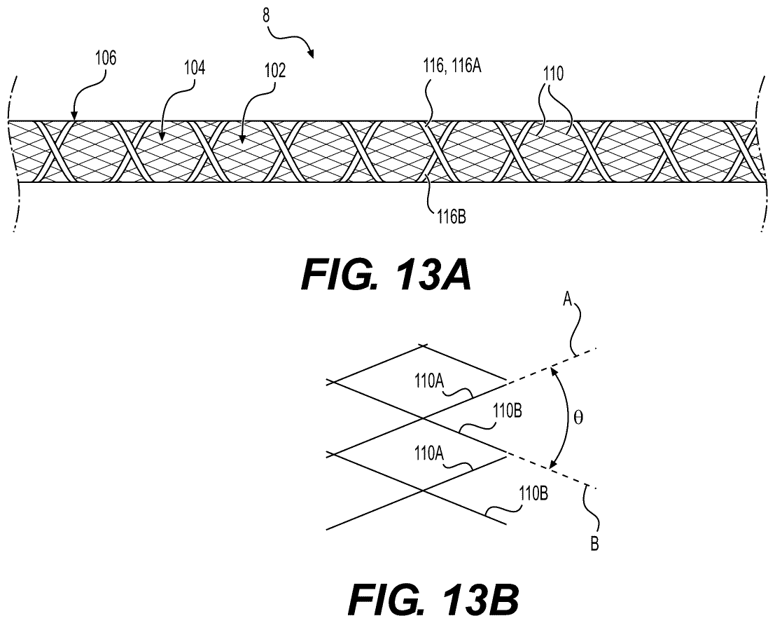

In some examples, the second layer 104 can be a braided layer. FIGS. 13A and 13B illustrate the sheath 8 with the outer layer 108 removed to expose the elastic third layer 106. With reference to FIGS. 13A and 13B, the braided second layer 104 can comprise a plurality of members or filaments 110 (for example, metallic or synthetic wires or fibers) braided together. The braided second layer 104 can have any desired number of filaments 110, which can be oriented and braided together along any suitable number of axes. For example, with reference to FIG. 13B, the filaments 110 can include a first set of filaments 110A oriented parallel to a first axis A, and a second set of filaments 110B oriented parallel to a second axis B. The filaments 110A and 110B can be braided together in a biaxial braid such that filaments 110A oriented along axis A form an angle θ with the filaments 110B oriented along axis B. In some examples, the angle θ can be from 5° to 70°, 10° to 60°, 10° to 50°, or 10° to 45°. In the illustrated example, the angle θ is 45°. In some examples, the filaments 110 can also be oriented along three axes and braided in a triaxial braid, or oriented along any number of axes and braided in any suitable braid pattern. The braided second layer 104 can extend along substantially the entire length L of the sheath 8, or alternatively, can extend only along a portion of the length of the sheath 8. In some examples, the filaments 110 can be wires made from metal (for example, Nitinol, stainless steel, etc.), or any of various polymers or polymer composite materials, such as carbon fiber. In some examples, the filaments 110 can be round, and can have a diameter of from 0.01 mm to 0.5 mm, 0.03 mm to 0.4 mm, or 0.05 mm to 0.25 mm. In some examples, the filaments 110 can have a flat cross-section with dimensions of 0.01 mm×0.01 mm to 0.5 mm×0.5 mm, or 0.05 mm×0.05 mm to 0.25 mm×0.25 mm. In one aspect, filaments 110 having a flat cross-section can have dimensions of 0.1 mm×0.2 mm. However, other geometries and sizes are also suitable for certain aspects. If braided wire is used, the braid density can be varied. Some examples have a braid density of from ten picks per inch to eighty picks per inch, and can include eight wires, sixteen wires, or up to fifty-two wires in various braid patterns. In some examples, the second layer 104 can be laser cut from a tube, or laser-cut, stamped, punched, etc., from sheet stock and rolled into a tubular configuration. The second layer 104 can also be woven or knitted, as desired.

The third layer 106 can be a resilient, elastic layer (also referred to as an elastic material layer). In some examples, the elastic third layer 106 can be configured to apply radially inward force to the underlying inner layer 102 and second layer 104 in a radial direction (for example, toward the central axis 114 of the sheath 8) when the sheath 8 expands beyond its natural diameter by passage of the delivery apparatus 10 through the sheath 8. Stated differently, the elastic third layer 106 can be configured to apply encircling/radially inward pressure to the layers of the sheath 8 beneath the elastic third layer 106 to counteract expansion of the sheath 8. The radially inwardly directed force is sufficient to cause the sheath 8 to collapse radially back to its unexpanded state after the delivery apparatus 10 is passed through the sheath 8.

In the illustrated example, the elastic third layer 106 can comprise one or more members configured as strands, ribbons, or elastic bands 116 helically wrapped around the braided second layer 104. For example, in the illustrated aspect the elastic third layer 106 comprises two elastic bands 116A and 116B wrapped around the braided second layer 104 with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some examples, the elastic layer can comprise an elastomeric material having a modulus of elasticity of 200 MPa or less. In some examples, the elastic third layer 106 can comprise a material exhibiting an elongation to break of 200% or greater, or an elongation to break of 400% or greater. The elastic third layer 106 can also take other forms, such as a tubular layer comprising an elastomeric material, a mesh, a shrinkable polymer layer such as a heat-shrink tubing layer, etc. In lieu of, or in addition to, the elastic third layer 106, the sheath 8 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In some examples, the elastic third layer 106 can also be radially outward of the polymeric outer layer 108.

In some examples, one or both of the inner layer 102 and/or the outer layer 108 can be configured to resist axial elongation of the sheath 8 when the sheath 8 expands. More particularly, one or both of the inner layer 102 and/or the outer layer 108 can resist stretching against longitudinal forces caused by friction between a prosthetic device (implant 12) and the inner surface of the sheath 8 such that the length L remains substantially constant as the sheath 8 expands and contracts. As used herein with reference to the length L of the sheath 8, the term “substantially constant” means that the length L of the sheath 8 increases by not more than 1%, by not more than 5%, by not more than 10%, by not more than 15%, or by not more than 20%. Meanwhile, with reference to FIG. 13B, the filaments 110A and 110B of the braided second layer 104 can be allowed to move angularly relative to each other such that the angle θ changes as the sheath 8 expands and contracts. This, in combination with the longitudinally-extending ridges 126 in the inner layer 102 and outer layer 108, can allow the lumen 112 of the sheath 8 to expand as a prosthetic device (for example, implant 12) is advanced through it.

For example, the inner layer 102 and the outer layer 108 can be heat-bonded during the manufacturing process such that the braided second layer 104 and the elastic third layer 106 are encapsulated between the inner layer 102 and outer layer 108. More specifically, in some examples the inner layer 102 and the outer layer 108 can be adhered to each other through the spaces between the filaments 110 of the braided second layer 104 and/or the spaces between the elastic bands 116. The inner layer 102 and outer layer 108 can also be bonded or adhered together at the proximal and/or distal ends of the sheath 8. In some examples, the inner layer 102 and outer layer 108 are not adhered to the filaments 110. This can allow the filaments 110 to move angularly relative to each other, and relative to the inner layer 102 and outer layer 108, allowing the diameter of the braided second layer 104, and thereby the diameter of the sheath 8, to increase or decrease. As the angle θ between the filaments 110A and 110B changes, the length of the braided second layer 104 can also change. For example, as the angle θ increases, the braided second layer 104 can shorten, and as the angle θ decreases, the braided second layer 104 can lengthen to the extent permitted by the areas where the inner layer 102 and outer layer 108 are bonded. However, because the braided second layer 104 is not adhered to the inner layer 102 and outer layer 108, the change in length of the braided layer that accompanies a change in the angle θ between the filaments 110A and 110B does not result in a significant change in the length L of the sheath 8.

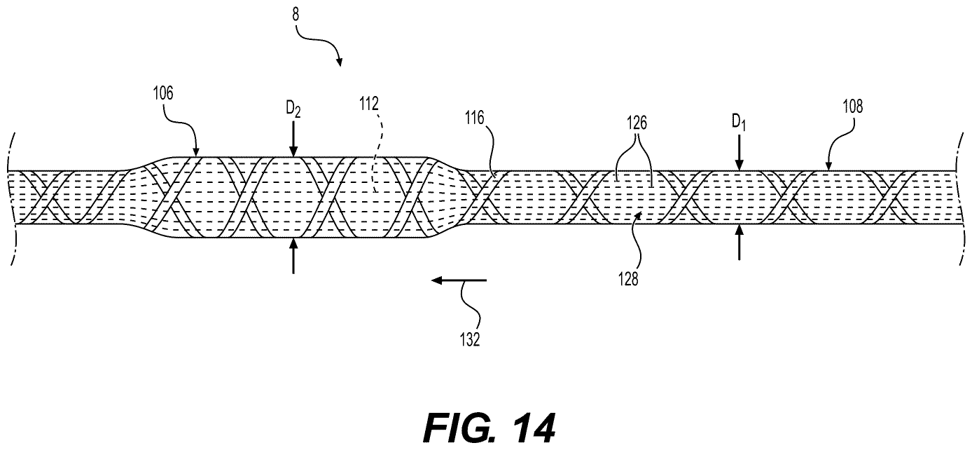

FIG. 14 illustrates radial expansion of the sheath 8 as a prosthetic device (for example, implant 12) is passed through the sheath 8 in the direction of arrow 132 (for example, distally). As the prosthetic device (for example, implant 12) is advanced through the sheath 8, the sheath 8 can resiliently expand to a second diameter D₂ that corresponds to a size or diameter of the prosthetic device (implant 12). As the prosthetic device (implant 12) is advanced through the sheath 8, the prosthetic device (implant 12) can apply longitudinal force to the sheath 8 in the direction of motion by virtue of the frictional contact between the prosthetic device (implant 12) and the inner surface of the sheath 8. However, as noted herein, the inner layer 102 and/or the outer layer 108 can resist axial elongation such that the length L of the sheath 8 remains constant, or substantially constant. This can reduce or prevent the braided layer second 104 from lengthening, and thereby constricting the lumen 112.

Meanwhile, the angle θ between the filaments 110A and 110B can increase as the sheath 8 expands to the second diameter D₂ to accommodate the implant 12 (for example, a prosthetic valve). This can cause the braided second layer 104 to shorten. However, because the filaments 110 are not engaged or adhered to the inner layer 102 or outer layer 108, the shortening of the braided second layer 104 attendant to an increase in the angle θ does not affect the overall length L of the sheath 8. Moreover, because of the longitudinally-extending ridges 126 formed in the inner layer 102 and outer layer 108, the inner layer 102 and outer layer 108 can expand to the second diameter D₂ without rupturing, in spite of being relatively thin and relatively non-elastic. In this manner, the sheath 8 can resiliently expand from its natural diameter D₁ to a second diameter D₂ that is larger than the diameter D₁ as a prosthetic device (implant 12) is advanced through the sheath 8, without lengthening, and without constricting. Thus, the force required to push the prosthetic implant 12 through the sheath 8 is significantly reduced.

Additionally, because of the radial force applied by the elastic third layer 106, the radial expansion of the sheath 8 can be localized to the specific portion of the sheath 8 occupied by the prosthetic device (implant 12). For example, with reference to FIG. 14, as the prosthetic device (for example, implant 12) moves distally through the sheath 8, the portion of the sheath 8 immediately proximal to the prosthetic device (implant 12) can radially collapse back to the initial diameter D₁ under the influence of the elastic third layer 106. The inner layer and outer layer 108 can also buckle as the circumference of the sheath 8 is reduced, causing the ridges 126 and the valleys 128 to reform. This can reduce the size of the sheath 8 required to introduce a prosthetic device (implant 12) of a given size. Additionally, the temporary, localized nature of the expansion can reduce trauma to the blood vessel into which the sheath 8 is inserted, along with the surrounding tissue, because only the portion of the sheath 8 occupied by the prosthetic device (implant 12) expands beyond the sheath's 8 natural diameter and the sheath 8 collapses back to the initial diameter once the device (implant 12) has passed. This limits the amount of tissue that must be stretched in order to introduce the prosthetic device (implant 12), and the amount of time for which a given portion of the vessel must be dilated.

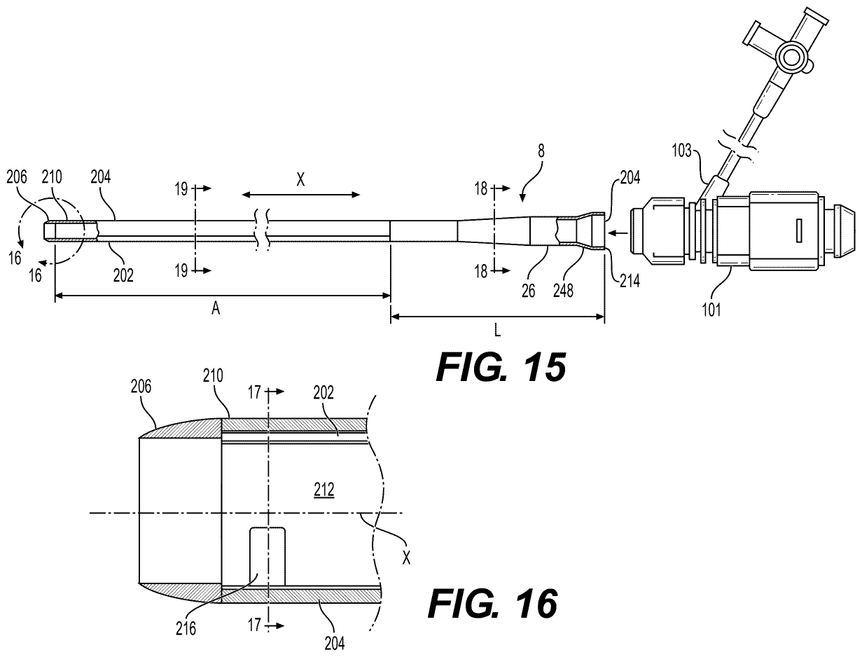

In some examples layered sheath 8 structure, FIGS. 15-23 illustrate various features of the coaxial layered structure of the expandable sheath 8 of FIG. 1 according to another aspect. Similar reference numbers are used to describe like elements. It is to be understood that the variations (for example, materials and alternate configurations) described herein with reference to FIGS. 11-14 can also apply to the example shown in FIGS. 15-23. Furthermore, the variations described herein with reference to FIGS. 15-23 can also be applied to the sheath 8 described in FIGS. 11-14.

Similar to various examples of the sheath 8 described herein in reference to FIGS. 11-14, the sheath 8 of FIGS. 15-23 includes a plurality of layers. For example, the sheath 8 illustrated in FIGS. 15-23, also includes an inner layer 202 and an outer layer 204 disposed around the inner layer 202. The inner layer 202 can define a central lumen 212 through which the delivery apparatus 10 travels into the patient's vessel in order to deliver, remove, repair, and/or replace a prosthetic device (implant 12), moving in a direction along the longitudinal axis X. Similar to the sheath 8 illustrated in FIGS. 11-14, as the prosthetic device (implant 12) passes through the sheath 8, the sheath 8 locally expands from a first, resting/unexpanded diameter to a second, expanded diameter to accommodate the prosthetic device (implant 12). After the prosthetic device (implant 12) passes through a particular location of the sheath 8, each successive expanded portion or segment of the sheath 8 at least partially returns to the smaller, resting/unexpanded diameter. In this manner, the sheath 8 can be considered self-expanding, in that it does not require use of a balloon, dilator, and/or obturator to expand.

Similar to the examples herein, the inner layer 202 and outer layer 204 can comprise any suitable materials. Suitable materials for the inner layer 202 include polytetrafluoroethylene (PTFE), ethylene tetrafluoroethylene (ETFE), nylon, polyethylene, polyether block amide (for example, Pebax), and/or combinations thereof. In an example sheath 8, the inner layer 202 can comprise a lubricious, low-friction, or hydrophilic material, such as PTFE. Such low coefficient of friction materials can facilitate passage of the prosthetic device (implant 12) through the lumen defined by the inner layer 202. In some examples, the inner layer 202 can have a coefficient of friction of less than about 0.1. Some examples of the sheath 8 can include a lubricious liner on the inner surface of the inner layer 202. Examples of suitable lubricious liners include materials that can further reduce the coefficient of friction of the inner layer 202, such as PTFE, polyethylene, polyvinylidene fluoride, and combinations thereof. Suitable materials for a lubricious liner also include other materials desirably having a coefficient of friction of about 0.1 or less.

Suitable materials for the outer layer 204 include nylon, polyethylene, Pebax, HDPE, polyurethanes (for example, Tecoflex), and other medical grade materials. In one example, the outer layer 204 can comprise high density polyethylene (HDPE) and Tecoflex (or other polyurethane material) extruded as a composite. In some examples, the Tecoflex can act as an adhesive between the inner layer 202 and the outer layer 204 and may only be present along a portion of the inner surface of the outer layer 204. Other suitable materials for the inner layer 202 and outer layer 204 are also disclosed in U.S. Pat. Nos. 8,690,936 and 8,790,387, which are incorporated herein by reference.

Additionally, some examples of the sheath 8 include an exterior hydrophilic coating on the outer surface of the outer layer 204. Such a hydrophilic coating can facilitate insertion of the sheath 8 into a patient's vessel. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (for example, PTFE, polyethylene, polyvinylidene fluoride), are also suitable for use with the sheath 8.

FIGS. 15-23 illustrate an example sheath system 200. FIG. 16 provides a partial cross-section of the distal end of the sheath 8 along section line 16-16 identified in FIG. 15. As described herein, the sheath 8 can be inserted into a vessel (for example, the femoral or iliac arteries) by passing through the skin of patient, such that a soft tip portion 206 at the distal end 210 of the sheath 8 is inserted into the vessel. As best seen in FIG. 16, the soft tip portion 206 can comprise, in some examples, low density polyethylene (LDPE) and can be configured to minimize trauma or damage to the patient's vessels as the sheath 8 is navigated through the vasculature. For example, the soft tip portion 206 can be slightly tapered to facilitate passage through the vessels. The soft tip portion 206 can be secured to the distal end 210 of the sheath 8, such as by thermally bonding the soft tip portion 206 to the inner layer 202 and outer layer 204 of the sheath 8. Such a soft tip portion 206 can be provided with a lower hardness than the other portions of the sheath 8. In some examples, the soft tip portion 206 can have a Shore hardness from about 25D to about 40D. The soft tip portion 206 is configured to be radially expandable to allow a prosthetic device (implant 12) to pass through the distal opening of the sheath 8. For example, the soft tip portion 206 can be formed with a weakened portion, such as an axially extending score line or perforated line that is configured to split and allow the soft tip portion 206 to expand radially when the prosthetic device passes (implant 12) therethrough.

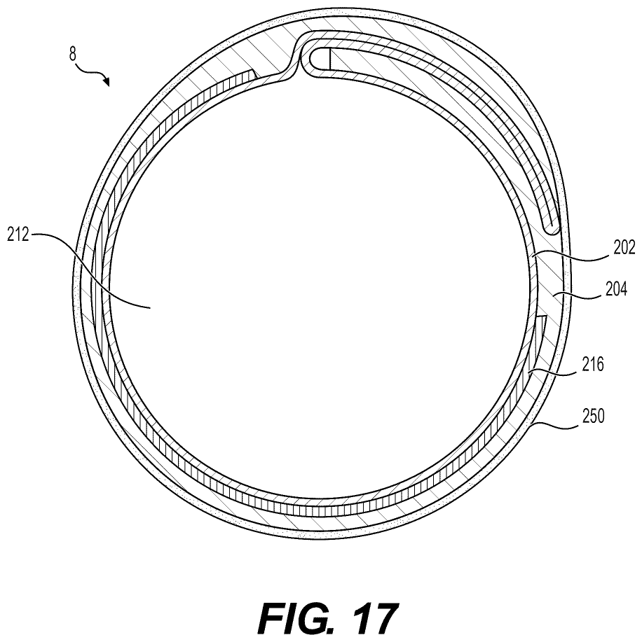

FIG. 17 shows a cross-sectional view of the sheath 8 taken near the distal end 210 of the sheath 8 as indicated by section line 17-17 in FIG. 16. As illustrated in FIGS. 16 and 17, the sheath 8 can include at least one radiopaque filler or marker, such as a discontinuous, or C-shaped, band/marker 216 positioned near the distal end 210 of the sheath 8. The marker 216 can be associated with the inner layer 202 and/or outer layer 204 of the sheath 8. For example, as shown in FIG. 17, the marker 216 can be positioned between the inner layer 202 and the outer layer 204. In some examples, the marker 216 can be associated with the outer surface of the outer layer 204. In some examples, the marker 216 can be embedded or blended within the inner layer 202 or outer layer 204.

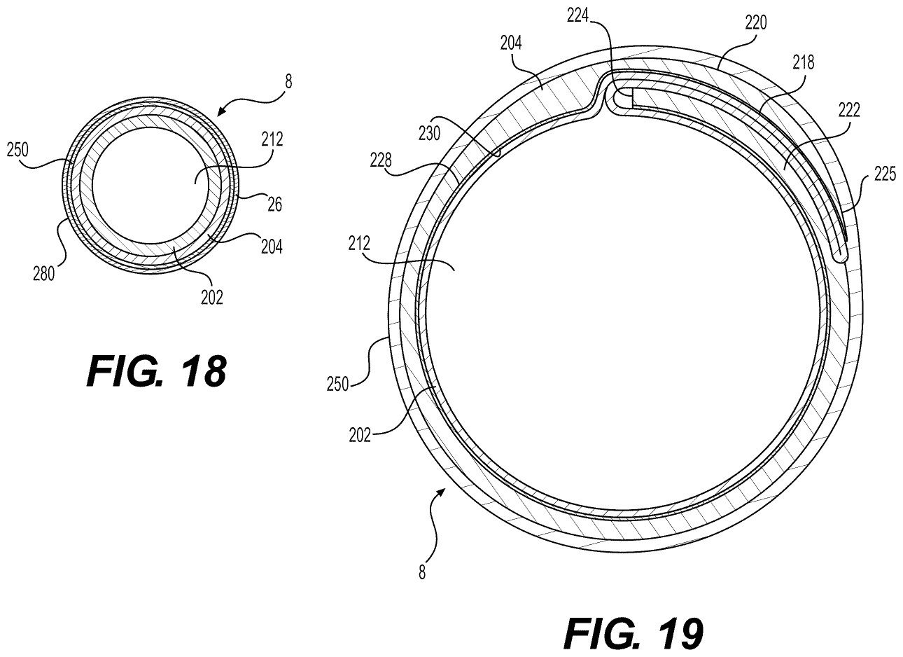

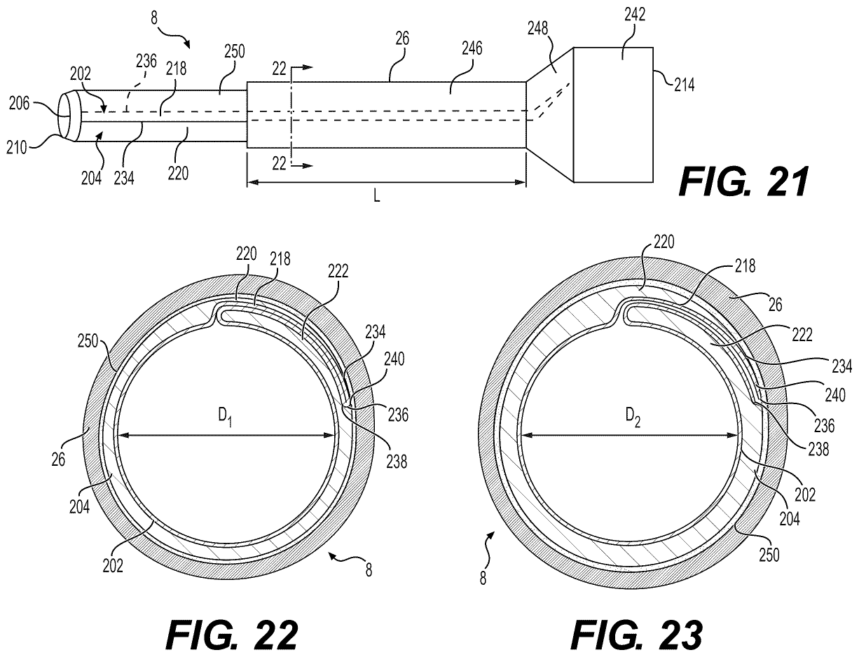

FIGS. 18 and 19 show additional cross-sections taken at different points along the sheath 8. FIG. 18 shows a cross-section of a segment of the sheath 8 near the proximal end 214 of the sheath 8, as indicated by section line 18-18 in FIG. 15. At this location, the sheath 8 includes the inner layer 202, outer layer 204, elastic outer layer 250/outer jacket, and the strain relief layer 26. At this location, near the proximal end 214 of the sheath 8, the inner layer 202 and outer layer 204 are substantially tubular. Here the inner layer 202 and outer layer 204 can be formed without any slits or folded portions in the layers. By contrast, as described herein, the inner layer and outer layer 204 at different locations along the sheath 8 (for example, at the point indicated by section line 19-19 in FIG. 15 and/or the point indicated by line section 22-22 in FIG. 21) can have a different configuration.

As shown in FIG. 19, the inner layer 202 can be arranged to form a substantially cylindrical central lumen 212 therethrough. Inner layer 202 can include one or more folded portions 218. In the example shown in FIG. 19, inner layer 202 is arranged to have one folded portion 218 that can be positioned on either side of the inner layer 202. Inner layer 202 can be continuous, in that there are no breaks, slits, or perforations in inner layer 202. Outer layer 204 can be arranged in an overlapping fashion such that an overlapping portion 220 overlaps at least a part of the folded portion 218 of the inner layer 202. As shown in FIG. 19, the overlapping portion 220 also overlaps an underlying portion 222 of the outer layer 204. The underlying portion 222 can be positioned to underlie both the overlapping portion 220 of the outer layer 204, as well as the folded portion 218 of the inner layer 202. Thus, the outer layer 204 can be discontinuous, in that it includes a slit or a cut in order to form the overlapping portion 220 and underlying portion 222. In other words, a first edge 224 of the outer layer 204 is spaced apart from a second edge 225 of the outer layer 204 so as not to form a continuous layer.

As shown in FIG. 19, the sheath 8 can also include a thin layer of bonding or adhesive material 228 positioned between the inner layer 202 and outer layer 204. In one example, the adhesive material 228 can comprise a polyurethane material such as Tecoflex. The adhesive material 228 can be positioned on an inner surface 230 of at least a portion of the outer layer 204 so as to provide adhesion between selected portions of the inner layer 202 and outer layer 204. For example, the outer layer 204 may only include a Tecoflex layer (adhesive material 228) around the portion of the inner surface 230 that faces the lumen-forming portion of the inner layer 202. In other words, the Tecoflex layer (adhesive material 228) can be positioned so that it does not contact the folded portion 218 of the inner layer 202 in some examples. In some examples, the Tecoflex can be positioned in different configurations as desired for the particular application. For example, as shown in FIG. 19, the Tecoflex layer can be positioned along the entire inner surface 230 of the outer layer 204. In some examples, the Tecoflex layer can be applied to the outer surface of the inner layer 202 instead of the inner surface of the outer layer 204. The Tecoflex layer can be applied to all or selected portions on the inner layer 202; for example, the Tecoflex layer can be formed only on the portion of the inner layer 202 that faces the lumen-forming portion of the outer layer 204 and not on the folded portion 218. The configuration of FIG. 19 allows for radial expansion of the sheath 8 as an outwardly directed radial force is applied from within (for example, by passing a medical device (implant 12) such as a prosthetic heart valve through the central lumen 212). As radial force is applied, the folded portion 218 can at least partially separate, straighten, and/or unfold, and/or the overlapping portion 220 and the underlying portion 222 of the outer layer 204 can slide circumferentially with respect to one another, thereby allowing the diameter of central lumen 212 to enlarge.

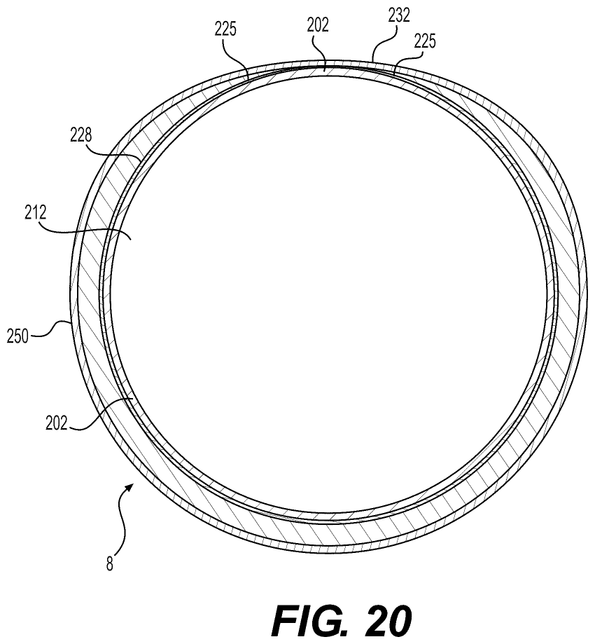

In this manner, the sheath 8 is configured to expand from a resting/unexpanded configuration (shown in FIG. 19) to an expanded configuration shown in FIG. 20. In the expanded configuration, as shown in FIG. 20, a gap 232 can form between the longitudinal edges of the overlapping portion 220 and the underlying portion 222 of the outer layer 204. As the sheath 8 expands at a particular location, the overlapping portion 220 of the outer layer 204 can move circumferentially with respect to the underlying portion 222 as the folded portion 218 of the inner layer 202 unfolds. This movement can be facilitated by the use of a low-friction material for inner layer 202, such as PTFE. Further, the folded portion 218 can at least partially separate and/or unfold to accommodate a medical device (implant 12) having a diameter larger than that of central lumen 212 in the resting/unexpanded configuration. As shown in FIG. 20, in some examples, the folded portion of the inner layer 202 can completely unfold, so that the inner layer 202 forms a cylindrical tube at the location of the expanded configuration.

Similar to the example sheath 8 in FIG. 14, the sheath 8 is configured to locally expands at a particular location corresponding to the location of the medical device (implant 12) along the length of the central lumen 212, and then locally contracts once the medical device (implant 12) has passed that particular location. Thus, a bulge may be visible, traveling longitudinally along the length of the sheath 8 as a medical device (for example, implant 12) is introduced through the sheath 8, representing continuous local expansion and contraction as the device travels the length of the sheath 8. Each segment of the sheath 8 will locally contract after removal of any radial outward force such that the sheath 8 at least partially returns to the original resting/unexpanded diameter of central lumen 212. Similar to the example sheath 8 described herein, an elastic outer layer 250 can optionally be provided along the sheath 8, urging the inner layer 202 and outer layer 204 back to the unexpanded configuration.

The inner layer 202 and outer layer 204 of sheath 8 can be configured having the folded portion 218 as shown in FIG. 19 along at least a portion of the length of the sheath 8. In some examples, the inner layer 202 and outer layer 204 can be configured as shown in FIG. 19 along the length A (FIG. 15) such that the folded portion 218 extends from a location adjacent the soft tip portion 206 to a location closer to the proximal end 214 of the sheath 8, adjacent and/or under the distal end of the strain relief layer 26. In this matter, the sheath 8 is expandable and contractable only along a portion of the length of the sheath 8 corresponding to length A (which can correspond to the section of the sheath 8 inserted into the narrowest section of the patient's vasculature).

In some examples, the folded portion 218 portion extends from a location adjacent the soft tip portion 206 under the strain relief layer 26, as illustrated in FIG. 21. In this example, the folded structure of the inner layer 202 extends from the soft tip portion 206, under the strain relief layer 26 and along the tapered portion 248 of the strain relief layer 26.

FIGS. 22 and 23 illustrate cross-sectional views of the sheath 8 taken along the strain relief layer 26 at section line 22-22 in FIG. 21. In this example, the folded portion 218 of the inner layer 202 extends under the strain relief layer 26. FIG. 22 shows a cross-section of the sheath 8 in a resting/unexpanded configuration having an inner diameter D₁. FIG. 23 shows a cross-section of the sheath 8 in a (partially) expanded configuration, having an inner diameter D₂, where D₂ is greater than D₁.

As shown in FIGS. 22-23, in some examples, the overlapping portion 220 does not overlap the entire folded portion 218 of the inner layer 202, and thus a portion of the folded portion 218 can be directly adjacent to the strain relief layer 26 in locations where the strain relief layer 26 is present. In locations where the strain relief layer 26 is not present, part of the folded portion 218 may be visible from the outside of the sheath 8, as seen in FIG. 21 (and/or visible through an elastic outer layer 250 described in more detail herein). In these examples, the sheath 8 can include a longitudinal seam 234 where the overlapping portion 220 terminates at the folded portion 218. In use, the sheath 8 can be positioned such that the seam 234 is posterior to the point of the sheath 8 that is 180 degrees from the seam 234 (for example, facing downward in the view of FIG. 21). As shown in FIG. 21, the seam 234 need not extend the entire length of the sheath 8, and end at a transition point between portions of the sheath 8 having a folded inner layer 202 and portions of the sheath 8 not having a folded inner layer 202.

In some examples, the folded portion 218 can include a weakened portion 236, such as a longitudinal perforation, score line, and/or slit, along at least a portion of the length of the inner layer 202. The weakened portion 236/slit allows for the two adjacent ends 238, 240 of the folded portion 218/inner layer 202 to move relative to one another as the sheath 8 expands to the expanded configuration shown in FIG. 23. For example, the sheath 8 locally expands as a medical device (for example, implant 12) is inserted therethrough, causing the weakened portion 236 to split/separate.

In each of the example sheaths 8 described herein, the sheath 8 may include an elastic outer layer 250 that expands with the sheath 8. The elastic outer layer 250 can provide an inwardly directed radial force that directs the sheath 8 to a folded/unexpanded configuration. Similar to the strain relief layer 26, elastic outer layer 250 can also provide hemostasis (for example, prevent blood loss during implantation of the prosthetic device, such as implant 12).

The elastic outer layer 250 can be positioned around at least a portion of the strain relief layer 26, outer layer 108, 204 and/or the inner layers 102, 202 of the sheath 8. As illustrated in FIGS. 21-23, the elastic outer layer 250 can surround the entire circumference of outer layer 204, and can extend longitudinally along any portion of the length of the sheath 8, including along (over or under) the strain relief layer 26. The elastic outer layer 250 extends for a length along at least a portion of the main body of the sheath 8. In some examples, the elastic outer layer 250 extends to a point adjacent the distal end 210, or can extend all the way to the distal end 210 of sheath 8. For example, the elastic outer layer 250 extends over the entire length of the sheath 8.

As shown in FIGS. 17-20, 22 and 23, the elastic outer layer 250 can be a continuous tubular layer, without slits or other discontinuities. The elastic outer layer 250 extends between strain relief layer 26 and the outer surface of the outer layer 204. In some examples, the elastic outer layer 250 extends over the outer surface of the strain relief layer 26 and the outer surface of the outer layer 204. In some examples, the elastic outer layer 250 extends both over the strain relief layer 26 and/or between the outer layer of the sheath 8 and the strain relief layer 26.

The elastic outer layer 250 can comprise any pliable, elastic material(s) that expand and contract, preferably with a high expansion ratio. Preferably, the materials used can include low durometer polymers with high elasticity, such as Pebax, polyurethane, silicone, and/or polyisoprene. Materials for the elastic outer layer 250 can be selected such that it does not impede expansion of the inner layer 202 and outer layer 204 of the sheath 8. The elastic outer layer 250 can have a thickness ranging from, for example, about 0.001 inches to about 0.010 inches. In some examples, the elastic outer layer 250 can have a thickness of about 0.003 inches to about 0.006 inches. The elastic outer layer 250 can be configured to stretch and expand as the sheath 8 expands, as shown in the expanded configuration in FIG. 20.

As illustrated in FIGS. 2, 15, 21 and 24, the sheath 8 in each of the examples described herein may include a strain relief layer 26. The strain relief layer 26 is provided adjacent the proximal end 214 of the sheath 8 and extends along/over the outer surface of the sheath 8. In some examples, the strain relief layer 26 is provided over the outer layer 108, 204 of the sheath 8. The strain relief layer 26 forms a smooth transition between the sheath hub 20 and the sheath 8 and facilitates mating of the sheath 8 with the sheath hub 20.

Additionally, and as will be described in more detail herein, the strain relief layer 26 provides a region of higher durometer or stiffness that restricts expansion of the underlying sheath layers. This helps to ensure hemostasis between the portions of the sheath 8 inside the patient and the sheath hub 20 (external to the patient). The increased durometer and/or stiffness along the strain relief layer 26 prevents blood from flowing between the various layers of the sheath 8 exterior to the patient during the procedure, helping to withstand the blood pressure that would otherwise cause the sheath 8 to “balloon up” with body fluid/blood. Additionally, the strain relief layer 26 can be sized and configured to form a seal with the patient's artery when inserted, such that blood is substantially prevented from flowing between the strain relief layer 26 and the vessel wall. For example, although the strain relief layer 26 does not extend all the way to the distal end 210 of the sheath 8, the strain relief layer 26 can extend distally enough along the sheath 8 that when the sheath 8 is fully inserted into the patient a portion of the strain relief layer 26 extends through and seals against the arteriotomy site.

As described herein, the strain relief layer 26 is provided over the outer layer 108, 204 of the sheath 8. The strain relief layer 26 can be bonded to the outer layer 108, 204 to prevent the strain relief layer 26 from sliding over the outer layer and “bunching up” in response to the friction forces applied by the surrounding tissue during insertion of the sheath 8 into the patient's vasculature. For example, the strain relief layer 26 can be bonded at the proximal end and/or distal end of the outer layer 108, 204. At the proximal and distal ends, the strain relief layer 26 can be bonded to the outer layer 204 around the full circumference of the outer layer. At the distal end of the sheath 8, the strain relief layer 26 can additionally and/or alternatively be bonded to the inner layer(s) of the sheath 8. For example, the strain relief layer 26 can be bonded to the distal end surface of the inner layer 102, 202.

FIGS. 18, 22 and 23 illustrate cross-sectional views of the sheath 8 along the strain relief layer 26. FIG. 18 shows a cross-section of a segment of the sheath 8 near the proximal end 214 of the sheath 8, as indicated by line 18-18 in FIG. 15. Similarly, FIGS. 22 and 23 show cross-section segments of various example sheaths 8 near the proximal end 214 of the sheath 8 and closer to the distal end of the strain relief layer 26, as indicated by section line 22-22 in FIG. 21. As illustrated in each of FIGS. 15-23, the sheath 8 at this location can comprise a liner/inner layer 202, outer layer 204, adhesive material 228, an optional elastic outer layer 250, and the strain relief layer 26.

The strain relief layer 26 extends circumferentially around at least a portion of the inner layer 202 and outer layer 204. The strain relief layer 26 extends from the proximal end 214 of the sheath 8 towards the distal end 210 of the sheath 8. As shown in FIG. 21 (and FIG. 15), the strain relief layer 26 extends for a length L along at least a portion of the main body of the sheath 8. In some examples, the strain relief layer 26 extends to a point adjacent the distal end 210, or can extend all the way to the distal end 210 of sheath 8. In some examples, the longitudinal length L of the strain relief layer 26 can range from about 10 cm to the entire length of the sheath 8.

The strain relief layer 26 extends to/adjacent the proximal end 214 of the sheath 8 and provides a compression fit over the distal end of the sheath hub 20 thereby coupling the sheath 8 to the sheath hub 20. Additionally, or alternatively, the strain relief layer 26 secured between the sheath hub 20 and the sheath hub cap 22 or other fastening device for by coupling the proximal end 214 of the sheath 8 to the sheath hub 20. In some examples, the strain relief layer 26 does not extend all the way to the proximal end 214 of the sheath 8.

It is understood that strain relief layer 26, as shown herein, can have similar composition and characteristics of the inner layer 102, 202 and outer layer 108, 204 as disclosed herein. Various compositions are disclosed, for example, in Application No. PCT/US2021/301275, entitled “Expandable sheath for introducing an endovascular delivery device into a body,” the disclosure of which is herein incorporated by reference.

The strain relief layer 26 can comprise any lubricious, low-friction, and/or relatively non-elastic material. Preferably the materials used can include high durometer polymers, with low elasticity. In some examples, the strain relief layer 26 is composed of the same and/or similar material to the inner layer 202 and/or outer layer 204. For example, as described herein regarding the inner layer 102 and/or outer layer 108, exemplary materials can include polyurethane (for example, high density polyethylene), ultra-high-molecular-weight polyethylene (UHMWPE) (for example, Dyneema®), high-molecular-weight polyethylene (HMWPE), or polyether ether ketone (PEEK). Other suitable materials strain relief layer 26 can include polyimide, polytetrafluoroethylene (PTFE), expanded polytetrafluoroethylene (ePTFE), ethylene tetrafluoroethylene (ETFE), nylon, polyethylene, polyamide, polyether block amide (for example, Pebax), and/or combinations of any of the herein. Materials for the strain relief layer 26 can be selected such that it impedes expansion of the underlying layers of the sheath 8.

The strain relief layer 26 can have a thickness ranging from, for example, about 0.001 inches to about 0.010 inches. In some examples, the strain relief layer 26 can have a thickness of from about 0.003 inches to about 0.006 inches. The wall thickness is measured radially between the inner surface of the strain relief layer 26 and the outer surface of the strain relief layer 26.

In some examples, the material composition and/or wall thickness can change along the length of the strain relief layer 26. For example, the strain relief layer 26 can be provided with one or more segments, where the composition and/or thickness changes from segment to segment. In an example aspect, the Durometer rating of the composition changes along the length of the strain relief layer 26 such that segments near the proximal end comprise a stiffer material or combination of materials, while segments near the distal end comprise a softer material or combination of materials. Similarly, the wall thickness of the strain relief layer 26 in segments near the proximal end can be thicker/greater than the wall thickness of the elastic outer layer 250 near the distal end.

As illustrated in FIGS. 15, 21 and 24, the strain relief layer 26 has a proximal end and a distal end and a central lumen 212 extending longitudinally therethrough. The strain relief layer 26 includes a generally tubular shaped proximal portion 242 adjacent the proximal end of the strain relief layer 26, and a generally tubular shaped distal portion 246 adjacent the distal end of the strain relief layer 26. The strain relief layer 26 includes a frustoconical shaped tapered portion 248 extending between the proximal portion 242 and the distal portion 246 of the strain relief layer 26, such that the diameter of the strain relief layer 26 at the proximal portion 242 is greater than the diameter of the strain relief layer 26 at the distal portion 246 of the strain relief layer 26. The tapered portion 248 and the flared proximal portion 242 help ease the transition of the medical device (implant 12)/delivery apparatus 10 when passing between the larger diameter sheath hub 20 to the smaller diameter of the sheath 8.

As described herein, the strain relief layer 26 is made of a material that is stiffer than the other sheath 8 layers such that the strain relief layer 26 inhibits expansion of the portion of the sheath 8 disposed along/under the strain relief layer 26. Because radial expansion is limited along the strain relief layer 26, higher push forces are necessary to advance the medical device (implant 12) through the central lumen 212 of the sheath 8. In some examples, the highest push forces through the sheath 8 are experienced near the proximal and distal ends of the sheath 8, through the strain relief layer 26 (for example, through the tapered portion of the strain relief layer 26), and at proximal and distal ends of the strain relief layer 26.

As described herein, it is desirable to reduce push forces required to insert the medical device (implant 12)/delivery apparatus 10 through the central lumen 212 of the sheath 8. In some examples, the thickness and/or composition of the strain relief layer 26 and/or sheath 8 can be adjusted to improve the performance of the sheath 8 and reduce the push force. In some examples, dilating or expanding the sheath 8 (or a portion thereof) before the medical device (implant 12)/delivery apparatus 10 is introduced helps to reduce the initial push force through the sheath 8. Pre-dilating the sheath 8 releases and/or loosens any bonding or adhesion of the sheath 8 layers that occurs during the manufacturing process, for example, bonding between the inner layer 202 and outer layer 204, bonding between the folded portion 218 and outer layer 204, bonding between the inner layer 202/outer layer 204 and the strain relief layer 26. Pre-dilating can also break or separate the weakened portion 236 of folded portion 218 of the inner layer 202, separating adjacent ends 238, 240 of the folded portion 218, as described herein and illustrated in FIG. 23. With the sheath 8 layers able to move freely with respect to the other, the medical device (implant 12)/delivery apparatus 10 is pushed through the sheath 8 lumen at a much lower force.

In some instances, the sheath 8 is pre-dilated by passing a relatively large dilator (for example, 22 French dilator) into the sheath 8 and through the strain relief layer 26. This can be done during sheath 8 preparation, prior to sheath 8 insertion into the patient and/or with the sheath 8 at least partially inserted into the patient. However, this method requires significant physical strength of the user (for example, grip and arm strength) to advance the dilator through the sheath 8, and particularly through the strain relief layer 26. Additionally, it is challenging to control the dilation distance. In some instances, it can be important that the sheath 8 not be dilated beyond the distal end of the strain relief layer 26. Expanding/dilating the sheath 8 beyond the end of the strain relief layer 26 can cause irregular sheath 8 expansion and difficulty or vessel injury during insertion, movement and/or withdraw of the sheath 8 in the vasculature. In some instances, it is desirable to dilate the distal opening of the sheath 8, a distal end portion of the sheath 8, and/or a length of the sheath 8 between the proximal and distal ends of the sheath 8 (for example, the portion of the sheath 8 including the strain relief layer 26). Current methods for controlling dilation of the sheath 8, including the rate, width/circumference, and longitudinal length, are prone to user error and inaccuracies because they rely on a user's visual observation of the dilator's movement within the sheath 8. For example, controlling dilation of the sheath 8 to only the portion including the strain relief layer 26 relied on visual observation of the dilator as it passes through the sheath 8/strain relief layer 26 and halting advancement of the dilator at the location where the portion of the sheath 8 beyond the distal end of the strain relief layer 26 starts to expand. Manual control of sheath 8 dilation is inherently difficult to train, difficult to enforce proper technique, and prone to errors.

In some examples, it is similarly desirable to dilate the patient's blood vessel proximate the treatment location and/or along the vessel route to the treatment location. For example, in some instances, the sheath 8, delivery apparatus 10 and/or medical device (implant 12) may encounter a narrowed or hardened blood vessel and/or a calcific lesion or other blockage/occlusion that impedes delivery of the sheath 8 or medical device (implant 12) to the treatment location. Traditional methods of widening the patient's blood vessel and/or dislodging a blockage involve inserting and withdrawing multiple and various sized dilators through the sheath 8 and into the patient's blood vessel. This multiple dilator technique increases the risk of trauma to the blood vessel and surrounding tissue, damage to the sheath 8, and increases the length of treatment time.

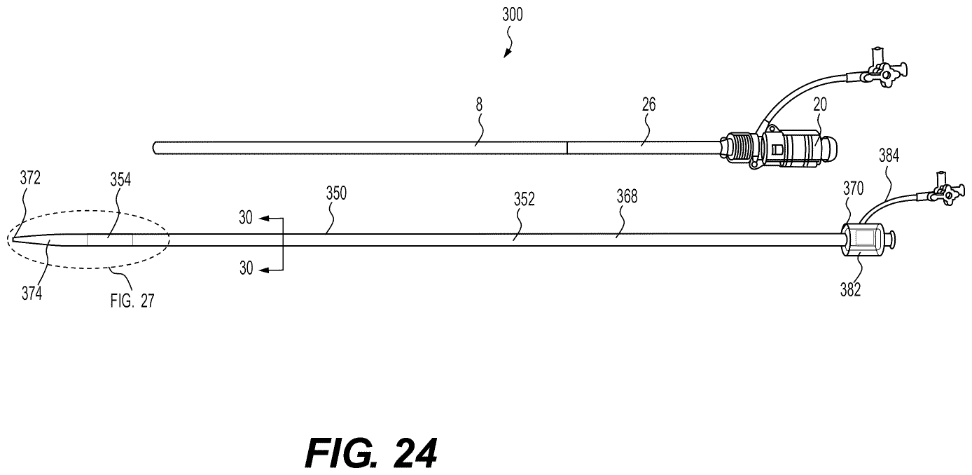

The present disclosure provides an example sheath system 300 that can be used to control the location, amount, and rate of expansion of a corresponding portion of the patient's blood vessel and/or sheath 8. The sheath system 300 is described in reference to FIGS. 24-32, and includes a combined introducer-dilator that is used to dilate/pre-dilate the blood vessel and/or sheath 8 in advance of medical device (implant 12) delivery. With the dilator 350 positioned within the sheath 8, the sheath system 300 provides sufficient column strength during sheath 8 insertion and advancement to the treatment site that multiple dilators and/or introducers are not necessary. As such, the dilator 350 provides for a combined introducer-dilator. The sheath system 300 also includes a variable diameter inflatable expansion element 354 that can be used to dilate the sheath 8, a tight or hardened blood vessel, and/or disrupt or dislodge a calcific lesion or other blockage/occlusion that impedes delivery of the sheath 8 and/or medical device (implant 12) to the treatment location.

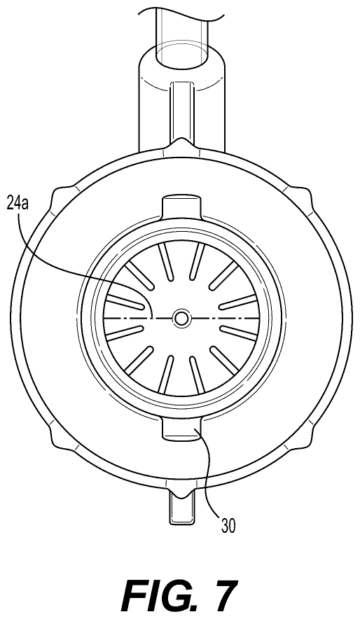

It is contemplated that the dilator 350 can be advanced within the central lumen of the sheath 8 described herein in reference to FIGS. 1-23. The dilator 350 can also be used individually and/or with another expandable sheath, including expandable sheaths having different layered sheath structures than those described herein.

FIG. 24 shows a side view of sheath system 300. The sheath system 300 includes a dilator 350 and a radially expandable sheath 8. For purposes of illustrations, the sheath system 300 will be described in reference to the expandable sheath 8 illustrated in FIGS. 11-14 and/or FIGS. 15-23. As described herein, in some examples, the sheath 8 includes a continuous inner layer (for example, inner layer 102, 202) defining a central lumen extending through the sheath 8, and a tubular strain relief layer 26 extending along the outer surface of the sheath 8 that limits radial expansion of the portion of the sheath 8 proximate the sheath hub 20. As described herein, the various layers of the sheath 8 and the strain relief layer 26 are configured to locally expand from an unexpanded configuration, at a first diameter, to an expanded configuration at a second, larger, diameter, due to the outwardly directed radial force exerted on the lumen of the sheath 8 (for example, inner layer 102, 202) by the dilator 350 and/or a medical device (implant 12), and then locally contract at least partially back to the unexpanded configuration as the dilator and/or medical device (implant 12) passes through the lumen.

FIGS. 24-26 show side views of the example dilator 350. The dilator 350 is similar to the introducer 6 provided in FIGS. 8A-8B and can optionally include an introducer locking hub 30 and or luer fitting at proximal end 370. The differences between the introducer 6 and the dilator 350 are provided in more detail herein.

The dilator 350 is sized and configured to be received within the central lumen of the sheath 8 and includes an elongated dilator shaft 352 with an inflatable expansion element 354 provided thereon. Similar to the introducer 6 (FIGS. 8A-8B), the dilator shaft 352, when received within the sheath 8, provides longitudinal and radial stiffness to the sheath 8 while also allowing sufficient flexibility for the combined sheath 8 and dilator 350 to be advanced through the patient's blood vessel to the treatment location. In some examples, the dilator 350/dilator shaft 352 has a Shore D durometer ranging from 63D to 75D. In some examples, the length of the dilator shaft 352 ranges from 25 cm to 100 cm. For example, in an example sheath system 300 used for femoral access, the length of the dilator shaft 352 is approximately 45 cm.

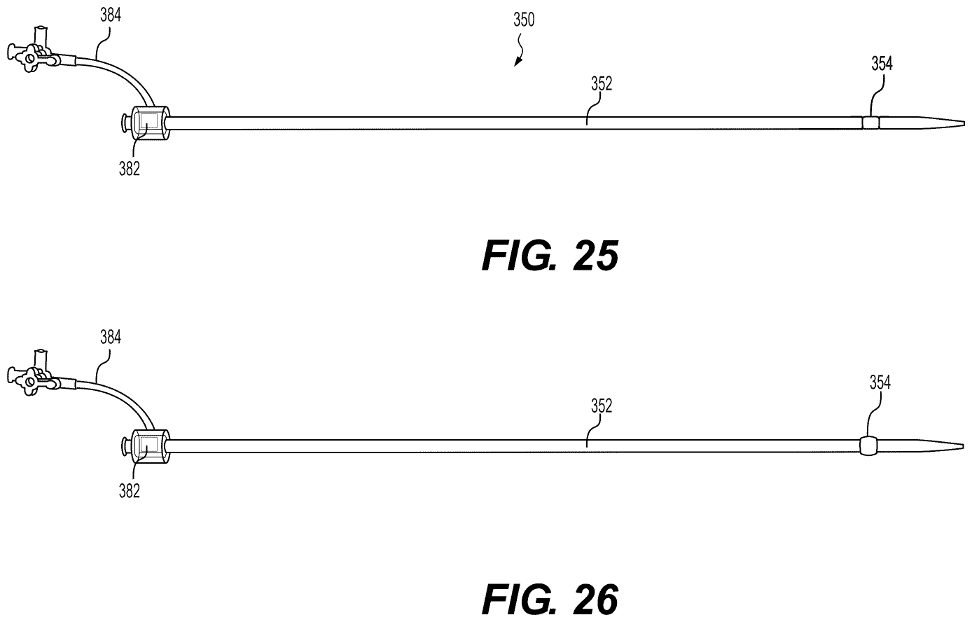

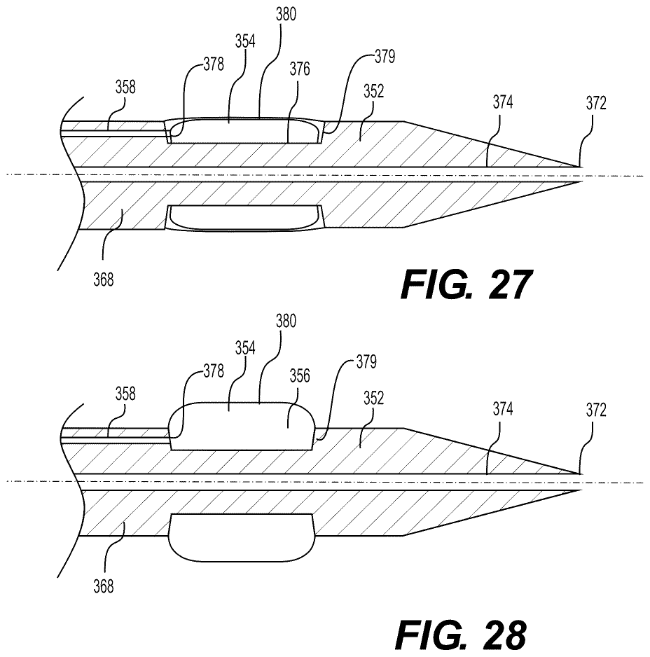

FIG. 25 illustrates a side view of the dilator 350 with the expansion element 354 in the unexpanded configuration, and FIG. 26 provides a corresponding side view of the dilator 350 with the expansion element 354 in the expanded configuration. Similarly, FIGS. 27 and 28 provide magnified views of the longitudinal cross-section of the distal end portion of the dilator 350 (as identified in FIG. 24) in the unexpanded and expanded configurations respectively.

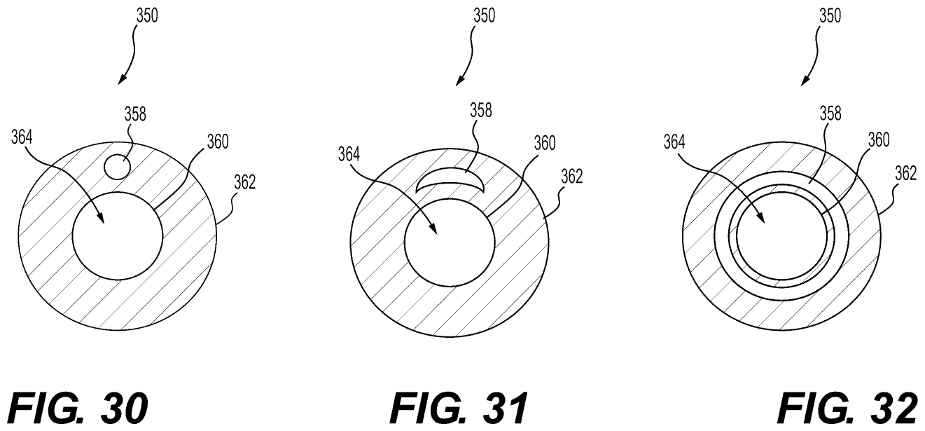

The dilator 350 includes a central lumen 364 extending through the dilator shaft 352, where the central lumen 364 defines the inner surface 360 of the dilator shaft 352 (see, for example, FIG. 30). In some examples, the central lumen 364 is sized for receiving a guidewire, for example, having a diameter of approximately 0.030 inches. In some examples, the inner surface 360/central lumen 364 is composed of a low friction material including for reducing friction between the dilator 350 and passing guidewire. In some examples, the low friction material includes HDPE or other fluoropolymers. In some examples, the inner surface 360/central lumen 364 includes a low friction coating, including, for example, hydrophilic coating or hydrophilically lubricious (polymer) blend material. Similarly, in some examples, the dilator 350, including the outer layer of the dilator 350, is composed of nylon 12.

As illustrated in FIGS. 25-28, the inflatable expansion element 354 is provided on the elongated body portion 368 of the dilator shaft 352 adjacent the distal end 372. The expansion element 354 encloses an inflation chamber 356 (for example, a fluid-bearing chamber) for receiving the inflation fluid. In some examples, the inflation fluid includes a fluid such as saline and/or a gas. The inflation fluid is provided to the inflation chamber 356 via an inflation lumen 358 in fluid communication with the inflation port 384 provided on the dilator hub 382. Upon receipt of the inflation fluid within the inflation chamber 356, the expansion element 354 is movable from the unexpanded configuration (FIGS. 25 and 27) to the expanded configuration (FIGS. 26 and 28). Similarly, withdrawal of the inflation fluid from the inflation chamber 356 causes the expansion element 354 to move from the expanded configuration back to the unexpanded configuration. In some examples, the volume of inflation fluid introduced into and/or withdrawn from the expansion element 354 is controlled, for example, by a mechanically or electrically controlled pump, including a computer controlled electrical pump.

As illustrated in FIGS. 25-28, in the unexpanded configuration the expansion element 354 has a first diameter, and in the expanded configuration the expansion element 354 has a second, larger diameter. Upon removal of the inflation fluid from the inflation chamber 356, the expansion element 354 returns to the unexpanded configuration and the first diameter. In some examples, in the expanded configuration, the expansion element 354 has a larger second diameter ranging from 12 F to 45 F. For example, in the expanded configuration, the larger second diameter of the expansion element 354 can range from 12 F (4 mm) to 45 F (15 mm), and the unexpanded first diameter can range from 9 F (3 mm) to 18 F (6 mm).

Similarly, in the unexpanded configuration, the inflation chamber 356 has a first volume and contains a first volume of inflation fluid, and in the expanded configuration the inflation chamber 356 has a larger second volume and contains a second, larger, volume of inflation fluid. Upon receipt of the inflation fluid into the inflation chamber 356, the expansion element 354 expands from the unexpanded configuration to the expanded configuration in response to the outwardly directed radial force exerted on the inner wall of the expansion element 354 from the increased volume of the inflation fluid received within the inflation chamber 356. Similarly, the expansion element 354 moves from the expanded configuration to the unexpanded configuration upon withdrawal of the inflation fluid from the inflation chamber 356 in response to a reduction in the outwardly directed radial force exerted on the inner wall of the expansion element 354 by the inflation fluid resulting from a decrease in the volume of the inflation fluid received within the inflation chamber 356.

As illustrated in FIGS. 26 and 28, in the expanded configuration, the expanded outer surface/perimeter of the expansion element 354 projects beyond the outer surface 362 of the dilator 350. In some examples, in the expanded configuration, the expansion element 354 projects beyond the outer surface 362 of the dilator 350 around the entire circumference of the dilator 350. In some examples, in the expanded configuration, the expansion element 354 projects beyond the outer surface 362 of the dilator 350 around a portion of the entire circumference of the dilator 350.

In some examples, in the expanded configuration, the expansion element 354 has any regular or irregular shaped cross-section (for example, radial and/or longitudinal cross-section). For example, the expanded expansion element 354 has a curvilinear shaped radial and/or longitudinal cross-section. In some examples, the expanded expansion element 354 has a circular shaped radial and/or longitudinal cross-section. In some examples, the expanded expansion element 354 has a rectilinear shaped radial and/or longitudinal cross-section. In some examples, as illustrated in FIG. 26, in the expanded configuration, the expansion element 354 has an elliptical shaped longitudinal cross-section.

In some examples, the expansion element 354 is composed of a semi-compliant (high-strength) material including, for example, Polyether Block Amide (Pebax), high durometer polyurethanes having Shore D durometer greater than 65D, polyamide. In some examples, the expansion element 354 is composed of a non-compliant (ultra high-strength) material including, for example, polyester such as polyethylene terephthalate or polybutylene terephthalate. In some examples, the expansion element 354 is composed of a polyamide, co-polyamide, polyether block amide, polyurethane (PE), polyolefin, polyethylene terephthalate (PET), nylon, or polybutylene terephthalate, or combinations thereof. In some examples, the expansion element 354 can resist burst pressure of at least about 5 atm.

As provided in FIG. 27, in some examples, when in the unexpanded configuration, the expansion element 354 is positioned against the outer diameter of the dilator 350 such that outer diameter of the unexpanded expansion element 354 is less than or equal to an outer diameter of the dilator 350. For example, when in the unexpanded configuration, the expansion element 354 is folded against the dilator shaft 352 such that the expansion element 354 does not extend beyond the outer diameter of the dilator 350. In some examples, when in the unexpanded configuration, the unexpanded expansion element 354 does not direct (or apply) radial force against in the central lumen of the blood vessel and/or sheath 8. In some examples, when in the unexpanded configuration, the expansion element 354 and/or the dilator shaft 352 can exert an outwardly directed radial force against the central lumen of the blood vessel and/or sheath 8.

As illustrated in FIG. 27, the dilator shaft 352 includes a reduced diameter portion 376 extending longitudinally/axially between the elongated body portion 368 of the dilator shaft 352 and the distal end portion 374. The elongated body portion 368 extends from the proximal end 370 of the dilator shaft 352 in a direction toward the distal end 372/reduced diameter portion 376. The distal end portion 374 extends from the distal end 372 in a direction toward the proximal end 370. In some examples, the distal end portion 374 includes a tapered surface extending from the distal end 372 of the dilator shaft 352 at an increasing taper toward the elongated body portion 368. The expansion element 354 can be compressed and/or folded against reduced diameter portion 376 of the dilator shaft 352. As such, the outer diameter of the of the unexpanded expansion element 354 is less than or equal to the outer diameter of the elongated body portion 368 of the dilator shaft 352. In some examples, in the unexpanded configuration, the expansion element 354 is folded against the reduced diameter portion 376. In some examples, the expansion element 354 is folded against the dilator shaft 352 in a propeller fold configuration. For example, in a propeller fold configuration, the expansion element 354 can optionally include longitudinally extending folded portions circumferentially flattened or pressed against the outer surface of the reduced diameter portion 376. In some examples, the expansion element 354 is folded against the dilator shaft 352 in a propeller fold configuration including five to eight folds.

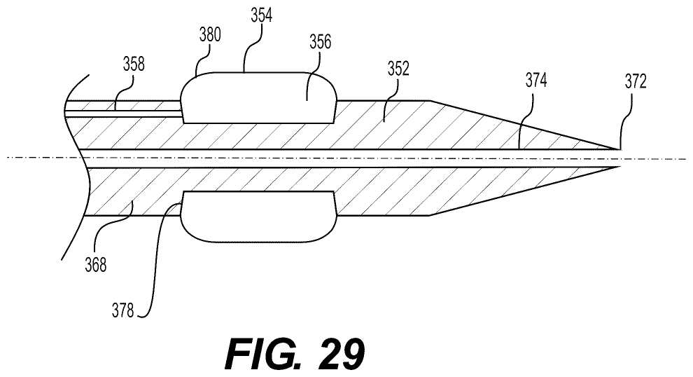

As provided in FIGS. 27 and 28, the reduced diameter portion 376 includes a proximally-tapered rear wall 378 and distally-tapered forward wall 379. In some examples, the rear wall 378 and forward wall 379 extend in a direction perpendicular to the longitudinal axis of the dilator shaft 352 and/or outer surface of the dilator shaft 352. FIG. 29 provides magnified view of the longitudinal cross-section of the distal end portion 374 of the dilator 350 of FIG. 24 according to some examples. As illustrated in FIG. 29, the reduced diameter portion 376 includes a distally-tapered rear wall 378, providing space for the unexpanded expansion element 354, thereby reducing the dilator 350 profile and helping with retrieval/removal from the patient's vasculature.

As illustrated in FIGS. 27-29, in some examples, the expansion element 354 is covered by an elastomer cover 380 that restrains the expansion element 354 in the unexpanded configuration and moves with the expansion element 354 during expansion. In some examples, the elastomer cover 380 has a low expansion force, for example, a Shore A durometer lower than 95A.

As described herein, inflation fluid is provided to the inflation chamber 356 via the inflation lumen 358. In some examples, the inflation lumen 358 is in fluid communication with an inflation port 384 provided on the dilator hub 382. The inflation lumen 358 can optionally be provided/extend between the inner surface 360/central lumen 364 and the outer surface 362 of the dilator 350. That is, the inflation lumen 358 extends longitudinally within the wall thickness of the dilator 350 defined between the inner surface 360 and the outer surface 362 of the dilator 350. The inflation lumen 358 can have any regular or irregular shaped cross-section. FIGS. 30-32 provide example cross-sectional shapes of the inflation lumen 358. For example, as illustrated in FIG. 30, the inflation lumen 358 has a circular shaped cross-section. In some examples, illustrated in FIG. 31, the inflation lumen 358 has a crescent shaped cross-section. In the example illustrated in FIG. 32, the inflation lumen 358 has an annular shaped cross-section.

A method of expanding a patient's blood vessel and/or the introducer sheath 8 to aid in delivering a medical device (for example, implant 12) to a treatment location is described herein. For illustrative purposes, the method is described in reference to the sheath 8 structure illustrated in FIGS. 15-23.

In some examples, the dilator 350 is advanced into the central lumen 212 of the sheath 8 with the expansion element 354 in the unexpanded configuration. For example, the dilator 350 is received within the central lumen 212 of the sheath 8 such that the unexpanded expansion element 354 is positioned within the central lumen 212. In some examples, the dilator 350 is received within the sheath 8 such that the distal end 372 of the dilator shaft 352 extends beyond the distal opening of the sheath 8, while the expansion element 354 remains within the central lumen 212. In some examples, the distal opening of the sheath 8 abuts the tapered surface of the distal end portion 374 of the dilator 350. In some examples, the dilator 350 is advanced further within the sheath 8 such that the distal opening of the sheath 8 abuts the cylindrical portion of the distal end portion 374.

If desired, the sheath 8 can be partially expanded before being inserted into the patient's blood vessel. For example, a first volume of inflation fluid is introduced into the inflation chamber 356 before and/or while inserted into the sheath 8, and before the sheath 8 is inserted at least partially into the blood vessel of the patient. The first volume of the inflation fluid at least partially expands the expansion element 354 which in turn at least partially expands a corresponding portion of the sheath 8 from the unexpanded configuration to the expanded configuration. Similarly, in some examples, the strain relief layer 26 of the sheath 8 is expanded before the sheath 8 is inserted and/or with the sheath 8 partially inserted into the patient's blood vessel.

With the dilator 350 received within the central lumen 212 of the sheath 8, the sheath 8 is inserted at least partially into the blood vessel of the patient. The sheath 8 is advanced within the blood vessel until the distal end of the sheath 8 is positioned at a location proximate the treatment site.

In some examples, the expansion element 354 can be used to expand the distal opening of the sheath 8. For example, the dilator 350 can be advanced within the central lumen 212 of the sheath 8 until the expansion element 354 is positioned at the desired location proximate the distal opening of the sheath 8. In some examples, when being used to expand the distal opening, the expansion element 354 extends at least partially through the distal opening of the sheath 8. The first volume of inflation fluid is introduced into the inflation chamber 356, expanding the distal opening. The first volume of inflation fluid can then be withdrawn from the expansion element 354.

As described herein, in some examples, the sheath system 300 is used to dilate the patient's blood vessel to reduce push forces required for delivering the sheath 8 and/or medical device (implant 12) to the treatment site. Accordingly, in some examples, the treatment site includes the delivery location of the medical device (implant 12), a narrowed or hardened blood vessel, tortuous vessel structure, and/or a calcific lesion or other blockage/occlusion that impedes delivery of the sheath 8/medical device (implant 12) to the treatment location. In this example, with the sheath 8 positioned at the treatment site, the dilator 350 is advanced within the central lumen 212 of the sheath and through the distal opening of the sheath 8 until the expansion element 354 is positioned at the desired treatment location within the patient's blood vessel. For example, the dilator 350 is advanced within the central lumen 212 of the sheath 8 until the expansion element 354 is located beyond the distal opening of the sheath 8 and within the narrowed or hardened portion of the blood vessel and/or adjacent any blockage/occlusion that impedes delivery of the sheath 8/medical device (implant 12). In some examples, where the sheath system 300 is used for mitral valve replacement/repair, the expansion element 354 can be located from 3 inches to 5 inches from the distal end 372 of the dilator shaft 352, allowing sufficient length for the cross-septal approach of the dilator shaft 352 and/or expansion element 354 between the left and right atrium.

With the expansion element 354 positioned at the treatment site, the inflation fluid is provided to the inflation chamber 356 thereby expanding the expansion element 354 from the unexpanded configuration to the expanded configuration. For example, inflation fluid is advanced through the inflation port 384, through the inflation lumen 358 and into the inflation chamber 356, expanding the expansion element 354. As the expansion element 354 expands, the adjacent portion of the patient's blood vessel expands in response to the outwardly directed radial force of the expansion element 354 against the vessel wall as the expansion element 354 moves from the unexpanded to the expanded configuration. As such, the expansion element 354 can be used to dilate the blood vessel before medical device (implant 12) delivery and/or advancement of the sheath 8 within the patient's vasculature thereby reducing the amount of push force required to advance the medical device (implant 12)/sheath 8. In some examples, expansion of the expansion element 354 is used to dilate a narrowed or hardened portion of the blood vessel in before the medical device (implant 12)/sheath 8 are delivered to the treatment location. In some examples, dilating the blood vessel will at least partially straighten tortious vessel structure. With the blood vessel dilated, the medical device (implant 12)/sheath 8 can pass through the vessel and to the delivery location of the medical device (implant 12) at a lower push force. In some examples, expansion of the expansion element 354 dislodges a blockage/occlusion (for example, calcific lesion) obstructing the blood vessel, thereby clearing the vessel and providing the medical device (implant 12)/sheath 8 access to the medical device delivery location.

The amount of dilation of the blood vessel and/or sheath 8 can be observed and controlled until the blood vessel (and/or sheath 8) reaches a desired dilation. For example, the amount of dilation can be observed using fluoroscopy or other imaging techniques. In some examples, the amount of dilation is observed and/or controlled by providing a pre-determined amount of inflation fluid to the expansion element 354, where the volume of the inflation fluid received within the expansion element 354 is associated with a corresponding volume and/or size (for example, diameter, length) of the expansion element 354.

With the blood vessel and/or sheath 8 dilated a desired amount, the inflation fluid is removed from the inflation chamber 356 and the expansion element 354 moves from the expanded configuration back to the unexpanded configuration. For example, the inflation fluid can be withdrawn from the inflation chamber 356 via the inflation lumen 358 and inflation port 384. In some examples, where the dilator 350 includes an elastomer cover 380, as the expansion element 354 moves back to the unexpanded configuration, the elastomer cover 380 can provide an inwardly directed radial force against the expansion element 354 urging it to the unexpanded configuration. In some examples, the elastomer cover 380 urges the expansion element 354 back into a folded configuration.

In some examples, the volume of inflation fluid introduced into and/or withdrawn from the expansion element 354 is controlled to vary the expansion of the expansion element 354 as desired. For example, the size and rate of expansion can be controlled based on patient anatomy, for example, initial and desired curvature of the blood vessel, initial and desired vessel diameter, relation between vessel size and implant size. In some examples, the volume of inflation fluid introduced into the expansion element 354 is controlled to direct the rate of expansion of the expansion element 354, for example, incrementally/gradually expand the blood vessel to reduce trauma and/or damage to the medical device (implant 12) or sheath 8.

With the expansion element 354 returned at least partially back to the unexpanded configuration, the dilator 350 is withdrawn from the central lumen 212 of the sheath 8. For example, the dilator 350 is moved proximally within the central lumen 212 toward the proximal end 214 of the sheath 8. In some examples, it is necessary that the expansion element 354 and/or dilator 350 be repositioned withing the sheath 8 or blood vessel. In which case, the dilator 350 is moved distally to reposition the expansion element 354 at the desired location and expanded.

To remove the dilator 350 from the sheath 8, the expansion element 354 is returned to the unexpanded configuration and withdrawn proximally from the sheath 8. With the dilator 350 removed from the sheath 8, the medical device (implant 12) and/or delivery apparatus 10 is introduced into the proximal end of the central lumen of the sheath 8. If the dilator 350 has been used to pre-dilate sheath 8 and/or strain relief layer 26, the push forces necessary to advance the medical device (implant 12)/delivery apparatus 10 through the sheath 8/strain relief layer 26 are reduced compared to a non-dilated sheath 8. Similarly, if the dilator 350 has been used to pre-dilate the patient's blood vessel, the push forces needed to advance the medical device/delivery apparatus 10 within the blood vessel and to the treatment site are reduced.

The medical device (implant 12) and delivery apparatus 10 can then be advanced into the sheath 8, particularly the portion of the sheath 8 including the strain relief layer 26. In some examples, the medical device (implant 12) is contracted or compressed radially as it passes through the strain relief layer 26, from the proximal portion 242, through the tapered portion 248 and into the smaller diameter distal portion 246.

As the medical device (implant 12) and delivery apparatus 10 are advanced through the strain relief layer 26, they exert an outwardly directed radial force against the central lumen of the sheath 8, causing the strain relief layer 26 (and corresponding portion of the sheath 8) proximate the medical device (implant 12)/delivery apparatus 10 to locally expand from an unexpanded configuration to an expanded configuration.

The medical device (implant 12) is then advanced beyond the distal end 27 of the strain relief layer 26, into the lumen of the sheath 8 beyond the strain relief layer 26. As the medical device (implant 12) is advanced through the sheath 8 beyond the strain relief layer 26, the sheath 8 locally expands from the unexpanded configuration to the expanded configuration at a location proximate the medical device (implant 12) in response to the outwardly directed radial force of the medical device (implant 12) exerted against the inner layer 202/central lumen 212 of the sheath 8. In some examples, the sheath 8 corresponds to the layered sheath 8 structure described in reference to FIGS. 11-23. The sheath 8 includes an inner layer 202 and/or outer layer 204, and a folded portion, for example, ridges 126 and valleys 128 of the fourth (outer) layer 108 (FIGS. 11-14), and folded portion 218 of the inner layer 202 (FIGS. 15-23). Locally expanding the lumen of the layered sheath 8 causes a length of the folded portion to at least partially unfold. Similarly, locally contracting the sheath 8 at least partially back to the unexpanded configuration causes a length of the folded portion to urge back to a folded configuration. For example, in reference to the sheath 8 of FIGS. 15-23, the outer layer is a discontinuous outer layer and includes an overlapping portion (for example, overlapping portion 220) and an underlying portion (for example, underlying portion 222). With the sheath 8 in the unexpanded configuration, the folded portion 218 is disposed between the overlapping portion 220 overlaps the underlying portion 222 (FIG. 19). As the sheath 8 locally expands to the expanded configuration, a length of the overlapping portion 220 moves circumferentially with respect to the underlying portion 220, at least partially unfolding the folded portion 218. As illustrated in FIG. 20, when the layered sheath 8 is fully expanded, the inner layer 202 extends into the gap 232 formed between the longitudinal edges of the overlapping portion 220 and the underlying portion 222 of the outer layer 204.

As the medical device (implant 12) passes through the lumen of the sheath 8/strain relief layer 26, the sheath 8/strain relief layer 26 locally contracts at least partially back to the unexpanded configuration when the medical device (implant 12) has passed. When used to deliver a medical device (implant 12) within a patient, the medical device (implant 12) is then passed through the distal opening of the sheath 8 and delivered to the treatment site. The position of the medical device (implant 12) can be moved or adjusted until the medical device (implant 12) is adequately positioned within the patient.

With the medical device (implant 12) delivered to the treatment site, any delivery apparatus 10/components coupled to the medical device (implant 12) are then removed from the medical device (implant 12) and withdrawn from/through the central lumen 212 of the sheath 8. In some examples, withdrawing the medical device from the central lumen 212 of the sheath 8 further includes locally contracting the sheath 8 at least partially back to the unexpanded configuration as the medical device (implant 12) passes through the central lumen 212, for example, by the radially inward force of an elastic outer layer 250/outer jacket provided over the sheath 8.

The sheath 8 is then withdrawn from the patient's blood vessel and the opening in the blood vessel and skin closed.

In some examples, the sheath 8 includes an elastic outer layer 250 that extends at least partially over the outer layer and/or the strain relief layer 26. The elastic outer layer 250 locally expands and contracts as the medical device (implant 12) is advanced through the lumen of the sheath 8. In some examples, the elastic outer layer 250 urges the various layers of the sheath 8 to an unexpanded configuration.

The medical device (for example, implant 12) described herein can include a prosthetic device mounted in a radially crimped state on a delivery apparatus (for example, delivery apparatus 10), and the act of advancing the prosthetic device through the lumen of the sheath 8 comprises advancing the delivery apparatus 10 and the prosthetic device through lumen of the sheath 8 and into the vasculature of the patient. In some examples, the prosthetic device comprises a prosthetic heart valve and the method further comprises implanting the prosthetic heart valve at a treatment site within the patient. In some examples, the prosthetic heart valve is mounted on a balloon catheter of the delivery apparatus 10 as the prosthetic heart valve is advanced through the sheath 8. In some examples, the sheath 8 is inserted into a femoral artery of the patient.

In view of the many possible examples to which the principles of the disclosed disclosure can be applied, it should be recognized that the illustrated examples are only preferred examples of the disclosure and should not be taken as limiting the scope of the disclosure. Rather, the scope of the disclosure is defined by the following claims. We, therefore, claim as our disclosure all that comes within the scope and spirit of these claims.

### Exemplary Aspects:

In view of the described processes and compositions, below are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: A sheath system comprising: a radially expandable sheath and a dilator sized and configured to be received within a central lumen of the sheath, the dilator including an expansion element provided thereon.

Example 2: The sheath system according to any example herein, particularly example 1, wherein the radially expandable sheath includes: a continuous inner layer defining the central lumen extending therethrough, the inner layer having at least one folded portion extending along a length of the inner layer; and the dilator sized and configured to be received within the central lumen of the sheath, the dilator including: an elongated dilator shaft including an expansion element provided thereon, the expansion element including an inflation chamber, wherein upon receipt of an inflation fluid within the inflation chamber, the expansion element is movable from an unexpanded configuration in which the expansion element has a first diameter to an expanded configuration in which the expansion element has a second, larger diameter, and upon removal of an inflation fluid from the inflation chamber the expansion element returns to the unexpanded configuration.

Example 3: The sheath system according to any example herein, particularly examples 1-2, further comprising an inflation fluid provided to the inflation chamber via an inflation lumen, wherein the expansion element expands from the unexpanded configuration to the expanded configuration upon receipt of the inflation fluid in the inflation chamber in response to an outwardly directed radial force exerted on the expansion element from an increase in a volume of the inflation fluid received within the inflation chamber, wherein the expansion element moves from the expanded configuration to the unexpanded configuration upon withdrawal of the inflation fluid from the inflation chamber in response a reduction in the outwardly directed radial force exerted on the expansion element by the inflation fluid resulting from a decrease in the volume of the inflation fluid received within the inflation chamber.

Example 4: The sheath system according to any example herein, particularly examples 1-3, wherein, in the unexpanded configuration, the expansion element is folded against the dilator shaft, wherein an outer diameter of the of the expansion element in the unexpanded configuration is less than or equal to an outer diameter of the dilator.

Example 5: The sheath system according to any example herein, particularly examples 1-4, wherein, the dilator shaft includes a reduced diameter portion, wherein the unexpanded configuration, the expansion element is folded against the reduced diameter portion such that the outer diameter of the expansion element is not greater than an outer diameter of the dilator shaft.

Example 6: The sheath system according to any example herein, particularly examples 1-5, wherein the dilator shaft includes an elongated body portion adjacent a proximal end of the dilator shaft, and a tapered portion extending from a distal end of the dilator shaft toward the body portion, wherein the expansion element is provided on the body portion proximate the distal end of the dilator shaft.

Example 7: The sheath system according to any example herein, particularly examples 1-6, wherein, when the dilator is advanced within the central lumen of the sheath, the expansion element is located beyond the distal end of the sheath.

Example 8: The sheath system according to any example herein, particularly examples 1-7, wherein the dilator further includes: an inner surface; an outer surface; a central lumen extending through the dilator, where the central lumen is defined by the inner surface of the dilator; and an inflation lumen in fluid communication with the inflation chamber for providing an inflation fluid thereto, where the inflation lumen is in fluid communication with an inflation port provided on a dilator hub coupled to a proximal end of the dilator, where the inflation lumen extends longitudinally within a wall thickness of the dilator provided between the inner surface and the outer surface of the dilator.

Example 9: The sheath system according to any example herein, particularly examples 1-8, wherein at least a portion of the sheath is configured to locally expand from an unexpanded configuration in which the central lumen has a first diameter to an expanded configuration in which the central lumen has a second, larger, diameter due to an outwardly directed radial force exerted on the central lumen of the inner layer by the dilator against the inner layer, and then locally contract at least partially back to the unexpanded configuration as the dilator and/or medical device passes through the central lumen.

Example 10: The sheath system according to any example herein, particularly examples 1-9, wherein the sheath further includes: an outer layer provided over the inner layer, where the outer layer is discontinuous and includes an overlapping portion and an underlying portion, and the overlapping portion overlaps the underlying portion, at least a portion of the folded portion of the inner layer is positioned between the overlapping an underlying portions, a tubular strain relief layer provided over the inner layer that limits radial expansion of the sheath, where the strain relief layer extends at least partially over the outer layer, wherein, when the sheath is in the unexpanded configuration, the at least one folded portion extends circumferentially over an outer surface of the inner layer and/or outer layer, wherein, when the sheath is in the expanded configuration, local expansion causes a length of the at least one folded portion to at least partially unfold and a length of the overlapping portion to move circumferentially with respect to the underlying portion.

Example 11. An inflatable dilator including an inflatable expansion element provided on the dilator shaft, the expansion element is movable from an unexpanded in which the expansion element has a first diameter to an expanded configuration in which the expansion element has a second, larger diameter.

Example 12. The inflatable dilator according to any example herein, particularly example 1, further including: an elongated dilator shaft sized and configured to be received within a central lumen of a sheath used for delivering a medical device; and the inflatable expansion element provided on the dilator shaft, the expansion element including an inflation chamber; wherein upon receipt of an inflation fluid within the inflation chamber, the expansion element is movable from the unexpanded configuration in which the expansion element has the first diameter to the expanded configuration in which the expansion element has the second, larger diameter, and upon removal of an inflation fluid from the inflation chamber the expansion element returns to the unexpanded configuration.

Example 13: The inflatable dilator according to any example herein, particularly examples 11-12, further including: an inflation fluid provided to the inflation chamber via an inflation lumen, wherein the expansion element expands from the unexpanded configuration to the expanded configuration upon receipt of the inflation fluid in the inflation chamber in response to an outwardly directed radial force exerted on the expansion element from an increase in a volume of the inflation fluid received within the inflation chamber, wherein the expansion element moves from the expanded configuration to the unexpanded configuration upon withdrawal of the inflation fluid from the inflation chamber in response a reduction in the outwardly directed radial force exerted on the expansion element by the inflation fluid resulting from a decrease in the volume of the inflation fluid received within the inflation chamber.

Example 14: The inflatable dilator according to any example herein, particularly examples 11-13, wherein the expansion element is composed of at least one of a semi-compliant (high-strength) material or a non-compliant (ultra high-strength) material.

Example 15: The inflatable dilator according to any example herein, particularly examples 11-14, wherein, in the unexpanded configuration, the expansion element is folded against the dilator shaft, wherein an outer diameter of the of the expansion element in the unexpanded configuration is less than or equal to an outer diameter of the inflatable dilator.

Example 16: The inflatable dilator according to any example herein, particularly examples 11-15, wherein, the dilator shaft includes a reduced diameter portion, wherein the unexpanded configuration, the expansion element is folded against the reduced diameter portion such that the outer diameter of the expansion element is not greater than an outer diameter of the dilator shaft.

Example 17: The inflatable dilator according to any example herein, particularly example 16, wherein the reduced diameter portion includes a distally-tapered rear wall.

Example 18: The inflatable dilator according to any example herein, particularly examples 11-17, wherein, in the unexpanded configuration, the expansion element is folded against the dilator shaft in a propeller fold configuration.

Example 19: The inflatable dilator according to any example herein, particularly examples 11-18, wherein the expansion element is covered by an elastomer cover.

Example 20. The inflatable dilator according to any example herein, particularly examples 11-19, wherein the dilator shaft includes an elongated body portion adjacent a proximal end of the dilator shaft, and a tapered portion extending from a distal end of the dilator shaft toward the body portion, wherein the expansion element is provided on the body portion proximate the distal end of the dilator shaft.

Example 21: The inflatable dilator according to any example herein, particularly examples 11-20, further including: an inner surface; an outer surface; a central lumen extending through the dilator, where the central lumen is defined by the inner surface of the dilator; and an inflation lumen in fluid communication with the inflation chamber for providing an inflation fluid thereto, where the inflation lumen is in fluid communication with an inflation port provided on a dilator hub coupled to the proximal end of the dilator, where inflation lumen extends longitudinally within a wall thickness of the dilator provided between the inner surface and the outer surface of the dilator.

Example 22: The inflatable dilator according to any example herein, particularly example 21, the inner surface includes a low friction material, a low friction coating, and/or a hydrophilic material or coating.

Example 23. A method of dilating a patient's blood vessel including: advancing a dilator within a patient's blood vessel and moving an inflatable expansion element provided on the dilator to an expanded configuration.

Example 24. A method of dilating a patient's blood vessel according to any example herein, particularly example 23 including: providing a radially expandable sheath including a continuous inner layer defining a central lumen therethrough, the inner layer having at least one folded portion extending along a length of the inner layer; advancing the dilator into the central lumen of the sheath with the inflatable expansion element provided on the dilator in an unexpanded configuration, the dilator including: an elongated dilator shaft with the expansion element including an inflation chamber provided thereon, wherein upon receipt of an inflation fluid within the inflation chamber, the expansion element is movable from the unexpanded configuration in which the expansion element has a first diameter to the expanded configuration in which the expansion element has a second, larger diameter, and upon removal of the inflation fluid from the inflation chamber the expansion element returns to the unexpanded configuration; inserting the sheath at least partially into the patient's blood vessel of the patient; advancing the dilator within the central lumen of the sheath and beyond a distal opening of the sheath until the expansion element is positioned at a treatment site within the patient's blood vessel; providing an inflation fluid to the inflation chamber thereby moving the expansion element from the unexpanded configuration to the expanded configuration and expanding the patient's blood vessel; removing the inflation fluid from the inflation chamber thereby moving the expansion element from the expanded configuration to the unexpanded configuration; and withdrawing the dilator from the central lumen of the sheath.

Example 25: A method of delivering a medical device through a sheath comprising: advancing a dilator within a central lumen of a radially expandable sheath; withdrawing the dilator from the central lumen of the sheath; and advancing the medical device through the central lumen of the sheath.

Example 26. A method of delivering a medical device through a sheath according to any example herein, particularly example 25, the method further comprising: providing the radially expandable sheath including a continuous inner layer defining the central lumen therethrough, the inner layer having at least one folded portion extending along a length of the inner layer; advancing the dilator into the central lumen of the sheath with an inflatable expansion element provided on the dilator in an unexpanded configuration, the dilator including: an elongated dilator shaft with the expansion element including an inflation chamber provided thereon, wherein upon receipt of an inflation fluid within the inflation chamber, the expansion element is movable from the unexpanded configuration in which the expansion element has a first diameter to an expanded configuration in which the expansion element has a second, larger diameter, and upon removal of the inflation fluid from the inflation chamber the expansion element returns to the unexpanded configuration; inserting the sheath and coupled dilator at least partially into a blood vessel of a patient; advancing the dilator within the central lumen of the sheath and beyond a distal opening of the sheath until the expansion element is positioned at a treatment site within the patient's blood vessel; providing an inflation fluid to the inflation chamber thereby moving the expansion element from the unexpanded configuration to the expanded configuration and expanding the patient's blood vessel; removing the inflation fluid from the inflation chamber thereby moving the expansion element from the expanded configuration to the unexpanded configuration; withdrawing the dilator from the central lumen of the sheath; advancing the medical device through the central lumen of the sheath causing the sheath to locally expand from the unexpanded configuration to the expanded configuration at a location proximate the medical device in response to an outwardly directed radial force of the medical device exerted against the inner layer and locally contracting the sheath at least partially back to the unexpanded configuration as the medical device passes through the central lumen; and advancing the medical device beyond the distal opening of the sheath to the treatment site.

Example 27: The method according to any example herein, particularly examples 25-26, wherein the inner layer includes at least one folded portion, wherein locally expanding the central lumen of the sheath causes a length of the folded portion to at least partially unfold.

Example 28: The method according to any example herein, particularly examples 25-27, wherein the sheath further includes: an outer layer provided over the inner layer, where the outer layer is discontinuous and includes an overlapping portion and an underlying portion, wherein when the sheath is in the unexpanded configuration, the overlapping portion overlaps the underlying portion with the folded portion of the inner layer disposed between the overlapping portion and the underlying portion, and a tubular strain relief layer provided over the inner layer that limits radial expansion of the sheath, where the strain relief layer extends at least partially over the outer layer.

Example 29: The method according to any example herein, particularly examples 25-28, wherein the medical device is a prosthetic device mounted in a radially crimped state on a delivery apparatus, wherein the prosthetic device comprises a prosthetic heart valve and the method further comprises implanting the prosthetic heart valve at a treatment site within the patient, wherein the prosthetic heart valve is mounted on a balloon catheter of a delivery apparatus as the prosthetic heart valve is advanced through the sheath.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

What is claimed is:

1. A sheath system comprising:
a radially expandable sheath including:
a continuous inner layer defining a central lumen extending therethrough, the inner layer having at least one folded portion extending along a length of the inner layer; and
a dilator sized and configured to be received within the central lumen of the sheath, the dilator including:
an elongated dilator shaft including an expansion element provided thereon, the expansion element including an inflation chamber,
wherein upon receipt of an inflation fluid within the inflation chamber, the expansion element is movable from an unexpanded configuration in which the expansion element has a first diameter to an expanded configuration in which the expansion element has a second, larger diameter, and upon removal of an inflation fluid from the inflation chamber the expansion element returns to the unexpanded configuration.

2. The sheath system of claim 1, further comprising an inflation fluid provided to the inflation chamber via an inflation lumen,
wherein the expansion element expands from the unexpanded configuration to the expanded configuration upon receipt of the inflation fluid in the inflation chamber in response to an outwardly directed radial force exerted on the expansion element from an increase in a volume of the inflation fluid received within the inflation chamber,
wherein the expansion element moves from the expanded configuration to the unexpanded configuration upon withdrawal of the inflation fluid from the inflation chamber in response a reduction in the outwardly directed radial force exerted on the expansion element by the inflation fluid resulting from a decrease in the volume of the inflation fluid received within the inflation chamber.

3. The sheath system of claim 1, wherein, in the unexpanded configuration, the expansion element is folded against the dilator shaft, wherein an outer diameter of the expansion element in the unexpanded configuration is less than or equal to an outer diameter of the dilator.

4. The sheath system of claim 1, wherein, the dilator shaft includes a reduced diameter portion, wherein the unexpanded configuration, the expansion element is folded against the reduced diameter portion such that an outer diameter of the expansion element is not greater than an outer diameter of the dilator shaft.

5. The sheath system of claim 1, wherein the dilator shaft includes an elongated body portion adjacent a proximal end of the dilator shaft, and a tapered portion extending from a distal end of the dilator shaft toward the body portion,
wherein the expansion element is provided on the body portion proximate the distal end of the dilator shaft.

6. The sheath system of claim 1, wherein, when the dilator is advanced within the central lumen of the sheath, the expansion element is located beyond a distal end of the sheath.

7. The sheath system of claim 1, wherein the dilator further includes:
an inner surface;
an outer surface;
a central lumen extending through the dilator, where the central lumen is defined by the inner surface of the dilator; and
an inflation lumen in fluid communication with the inflation chamber for providing an inflation fluid thereto, where the inflation lumen is in fluid communication with an inflation port provided on a dilator hub coupled to a proximal end of the dilator, where the inflation lumen extends longitudinally within a wall thickness of the dilator provided between the inner surface and the outer surface of the dilator.

8. The sheath system of claim 1, wherein at least a portion of the sheath is configured to locally expand from an unexpanded configuration in which the central lumen has a first diameter to an expanded configuration in which the central lumen has a second, larger, diameter due to an outwardly directed radial force exerted on the central lumen of the inner layer by the dilator against the inner layer, and then locally contract at least partially back to the unexpanded configuration as the dilator and/or medical device passes through the central lumen.

9. The sheath system of claim 1, wherein the sheath further includes:
an outer layer provided over the inner layer, where the outer layer is discontinuous and includes an overlapping portion and an underlying portion, and the overlapping portion overlaps the underlying portion, at least a portion of the folded portion of the inner layer is positioned between the overlapping an underlying portions, and
a tubular strain relief layer provided over the inner layer that limits radial expansion of the sheath, where the strain relief layer extends at least partially over the outer layer,
wherein, when the sheath is in the unexpanded configuration, the at least one folded portion extends circumferentially over an outer surface of the inner layer and/or outer layer,
wherein, when the sheath is in the expanded configuration, local expansion causes a length of the at least one folded portion to at least partially unfold and a length of the overlapping portion to move circumferentially with respect to the underlying portion.

10. An inflatable dilator including:
an elongated dilator shaft sized and configured to be received within a central lumen of a sheath used for delivering a medical device; and
an inflatable expansion element provided on the dilator shaft, the expansion element including an inflation chamber;
wherein upon receipt of an inflation fluid within the inflation chamber, the expansion element is movable from an unexpanded in which the expansion element has a first diameter to an expanded configuration in which the expansion element has a second, larger diameter, and upon removal of an inflation fluid from the inflation chamber the expansion element returns to the unexpanded configuration.

11. The inflatable dilator of claim 10, further including:
an inflation fluid provided to the inflation chamber via an inflation lumen,
wherein the expansion element expands from the unexpanded configuration to the expanded configuration upon receipt of the inflation fluid in the inflation chamber in response to an outwardly directed radial force exerted on the expansion element from an increase in a volume of the inflation fluid received within the inflation chamber,
wherein the expansion element moves from the expanded configuration to the unexpanded configuration upon withdrawal of the inflation fluid from the inflation chamber in response a reduction in the outwardly directed radial force exerted on the expansion element by the inflation fluid resulting from a decrease in the volume of the inflation fluid received within the inflation chamber.

12. The inflatable dilator of claim 10, wherein the expansion element is composed of at least one of a semi-compliant material or a non-compliant material.

13. The inflatable dilator of claim 10, wherein, in the unexpanded configuration, the expansion element is folded against the dilator shaft, wherein an outer diameter of the of the expansion element in the unexpanded configuration is less than or equal to an outer diameter of the inflatable dilator.

14. The inflatable dilator of claim 10, wherein, the dilator shaft includes a reduced diameter portion, wherein the unexpanded configuration, the expansion element is folded against the reduced diameter portion such that an outer diameter of the expansion element is not greater than an outer diameter of the dilator shaft.

15. The inflatable dilator of claim 14, wherein the reduced diameter portion includes a distally-tapered rear wall.

16. The inflatable dilator of claim 10, wherein, in the unexpanded configuration, the expansion element is folded against the dilator shaft in a propeller fold configuration.

17. The inflatable dilator of claim 10, wherein the expansion element is covered by an elastomer cover.

18. The inflatable dilator of claim 10, wherein the dilator shaft includes an elongated body portion adjacent a proximal end of the dilator shaft, and a tapered portion extending from a distal end of the dilator shaft toward the body portion,
wherein the expansion element is provided on the body portion proximate the distal end of the dilator shaft.

19. The inflatable dilator of claim 10, further including:
an inner surface;
an outer surface;
a central lumen extending through the dilator, where the central lumen is defined by the inner surface of the dilator; and
an inflation lumen in fluid communication with the inflation chamber for providing an inflation fluid thereto, where the inflation lumen is in fluid communication with an inflation port provided on a dilator hub coupled to a proximal end of the dilator, where inflation lumen extends longitudinally within a wall thickness of the dilator provided between the inner surface and the outer surface of the dilator.

20. A method of dilating a patient blood vessel including:
providing a radially expandable sheath including a continuous inner layer defining a central lumen therethrough, the inner layer having at least one folded portion extending along a length of the inner layer;
advancing a dilator into the central lumen of the sheath with an inflatable expansion element provided on the dilator in an unexpanded configuration, the dilator including:
an elongated dilator shaft with the expansion element including an inflation chamber provided thereon, wherein upon receipt of an inflation fluid within the inflation chamber, the expansion element is movable from the unexpanded configuration in which the expansion element has a first diameter to an expanded configuration in which the expansion element has a second, larger diameter, and upon removal of the inflation fluid from the inflation chamber the expansion element returns to the unexpanded configuration;
inserting the sheath at least partially into the patient's blood vessel of the patient;
advancing the dilator within the central lumen of the sheath and beyond a distal opening of the sheath until the expansion element is positioned at a treatment site within the patient's blood vessel;
providing an inflation fluid to the inflation chamber thereby moving the expansion element from the unexpanded configuration to the expanded configuration and expanding the patient's blood vessel;
removing the inflation fluid from the inflation chamber thereby moving the expansion element from the expanded configuration to the unexpanded configuration; and
withdrawing the dilator from the central lumen of the sheath.
